(19) United States
(12) Patent Application Publication
(10) Pub. No.: US 2024/0000885 A1
(43) Pub. Date: Jan. 4, 2024

(54) COMPOSITIONS AND METHODS FOR TARGETING THE INTERACTION OF HOST MYO5B+D AND CORONAVIRUS M PROTEINS

(71) Applicants: VANDERBILT UNIVERSITY, Nashville, TN (US); UNIVERSITEIT UTRECHT HOLDING B.V., utrecht (NL)

(72) Inventors: Frédéric TANGY, Les Lilsa (FR); Pierre-Olivier VIDALAIN, Lyon (FR); James R. GOLDENRING, Nashville, TN (US); Joseph T. Roland, Nashville, TN (US); Lynne A. LAPIERRE, Nashville, TN (US); Elizabeth MANNING, Nashville, TN (US); Cornelis Alexander Maria DE HAAN, Utrecht (NL)

(21) Appl. No.: 18/251,497

(22) PCT Filed: Nov. 3, 2021
(86) PCT No.: PCT/US2021/072201

§ 371 Date: May 2, 2023

Related U.S. Application Data
(60) Provisional application No. 63/228,384, filed on Aug. 2, 2021.
(60) Provisional application No. 63/109,941, filed on Nov. 5, 2020.

(51) Int. Cl. A61K 38/16; A61K 31/426; A61P 31/04; C07K 16/18

(52) U.S. Cl. CPC A61K 38/162; C07K 16/18; A61P 31/04; A61K 31/426

(57) ABSTRACT

Disclosed herein is a method for treating or preventing a coronavirus infection in a subject that involves administering to the subject an effective amount of a composition comprising an agent that disrupts the binding of coronavirus membrane glycoprotein (M protein) and Myosin Vb protein (MYO5B). Also disclosed herein is a method for identifying an agent for treating or preventing a coronavirus infection that involves providing a system comprising coronavirus membrane glycoprotein (M protein) and human Myosin Vb protein (MYO5B) with conditions suitable for binding of the M protein and MYO5B; contacting the system with a candidate agent; and assaying the system for binding of M protein and MYO5B.

### CROSS-REFERENCE TO RELATED APPLICATIONS

This application claims benefit of U.S. Provisional Application No. 63/109,941, filed Nov. 5, 2020, and U.S. Provisional Application No. 63/228,384, filed Aug. 2, 2021, which are hereby incorporated herein by reference in their entireties.

### STATEMENT REGARDING FEDERALLY SPONSORED RESEARCH OR DEVELOPMENT

This invention was made with Government Support under Grant No. NSF2032016 awarded by the National Science Foundation, and Grant No. DK48370 awarded by the National Institutes of Health. The Government has certain rights in the invention.

### SEQUENCE LISTING

This application contains a sequence listing filed in electronic form as an ASCII.txt file entitled “222230-2030 Sequence Listing_ST25” created on Oct. 27, 2021 and having 32,167 bytes. The content of the sequence listing is incorporated herein in its entirety.

### BACKGROUND

The coronavirus family includes multiple pathogenic viruses including Mouse Hepatitis Virus (MHV), Porcine Epidemic Diarrhea Virus (PEDV), MERS, SARS and SARS-CoV-2 (COVID-19) as well as the common cold virus (Cui, J., et al. Nature reviews. Microbiology 2019 17:181-192). Compositions and methods are needed to treat or prevent the effects of coronavirus infection.

### SUMMARY

The membrane glycoprotein (M protein) of coronaviruses serves as the nidus for the assembly of the coronavirus virion. Nevertheless, relatively little is known concerning interactions with host proteins that may mediate trafficking through host cell organelles. Using a yeast two-hybrid, Myosin Vb (MYO5B) was identified as interacting with the cytosolic tail of MHV M protein. Mapping demonstrated that MHV M interacts specifically with the alternatively spliced Exon D of MYO5B (MYO5B+D) that is expressed in lung, heart, brain and intestine and mediates interaction with Rab10. MYO5B+D co-localized with MHV M protein when co-expressed in MDCK cells. MYO5B+D also co-localized with M proteins from MERS, PEDV and SARS-CoV-2 when co-expressed in MDCK cells. Co-localization was observed with endogenous Rab10 and Rab11a. Similar results were also observed in co-expression studies in A549 lung cells. Point mutations were identified in MHV M that blocked the interaction with MYO5B+D in both yeast 2-hybrid assays and co-localization in MDCK and A549 cells. One of these point mutations (E121K) has previously been shown to block MHV virion assembly. The disclosed results suggest that the interaction of coronavirus M proteins with MYO5B+D may regulate appropriate trafficking of M proteins in host cells through interactions within plasma membrane recycling systems in polarized epithelial cells.

Therefore, disclosed herein is a method for treating or preventing a coronavirus infection in a subject that involves administering to the subject an effective amount of a composition comprising an agent that disrupts the binding of coronavirus membrane glycoprotein (M protein) and Myosin Vb protein (MYO5B).

In some embodiments, the agent is an antibody or aptamer that selectively binds M protein at or near its MYO5B binding site, or selectively binds MYO5B at or near its M protein binding site.

In some embodiments, the agent is a small molecule, such as a molecule identified by a screening method described herein.

In some embodiments, the agent is a soluble fragment of M protein capable of binding human MYO5B. In some embodiments, the agent is a soluble fragment of MYO5B capable of binding M protein. In particular, the agent can be a soluble fragment of MYO5B comprising exons ABCDE of MYO5B containing Exon D (MYO5B+D).

Also disclosed herein is a method for identifying an agent for treating or preventing a coronavirus infection that involves providing a system comprising coronavirus membrane glycoprotein (M protein) and human Myosin Vb protein (MYO5B) with conditions suitable for binding of the M protein and MYO5B; contacting the system with a candidate agent; and assaying the system for binding of M protein and MYO5B. In these methods an inhibition in M protein and MYO5B binding is an indication that the agent can be used to treat or prevent a coronavirus infection.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1D show the MHV M cytoplasmic tail interacts with MYO5B+D. FIG. 1A shows yeast 2-hybrid interactions between pAD-Myosin Vb targets and various interacting proteins including Rab GTPases and Rab11-FIP2 which are known to interact with Myosin V proteins. Note that the MHV M cytoplasmic carboxy-terminal tail only interacts with Myosin Vb constructs that include the D exon. FIG. 1B shows truncation and random mutagenesis reveals requisite binding regions in MHV M tail (amino acids 105-228) . Truncations of MHV M cytoplasmic tail or random mutagenesis of pBD-MHV M tail were assayed in yeast 2-hybrid reactions with pAD-MYO5B(ABCDE). Yeast 2-hybrid results are noted at the right (Y2H). FIG. 10 shows co-expression MHV M-GFP with Cherry-MYO5B+D or Cherry MYO5BΔD and immunostaining of endogenous Rab11a in MDCK cells. Z axis projections are shown below X-Y slice images, with the merged overlap at the right. FIG. 1D shows co-expression of MHV M-GFP with Cherry MYO5B+D and endogenous Rab10 immunostaining in MDCK cells. All results are representative of 4 separate experiments. Bar=5 μm.

FIGS. 2A and 2B show point mutants in MHV M cytoplasmic tail block co-localization with co-expressed Cherry-MYO5B+D. MHV M-GFP mutants for E121K (FIG. 2A) M126T (FIG. 2B) were expressed alone or with Cherry-MYO5B+D in MDCK cells. All cells were immunostained for endogenous Rab10. Z axis projections are shown below X-Y slice images, with the merged overlap at the right. Bar=5 μm. Results are representative of 4 individual experiments.

FIGS. 3A to 3C show co-localization of coronavirus M proteins with co-expressed MYO5B+D. GFP chimeras of PEDV (FIG. 3A), MERS (FIG. 3B), and SARS-CoV-2 (FIG. 3C) M proteins were expressed with either Cherry-MYO5B+D or Cherry-MYO5BΔD in MDCK cells. Cells were immunostained for endogenous Rabila and F-Actin (Phalloidin). Z axis projections are shown below X-Y slice images, with the merged overlap at the right. Bar=5 μm. Results are representative of 4 individual experiments.

FIGS. 4A to 4C show loss of co-localization with Cherry-MYO5B+D with point mutations in coronavirus M proteins. GFP chimeras of PEDV(E115K) (FIG. 4A), MERS(E114K) (FIG. 4B), and SARS-CoV-2(E115K) (FIG. 4C) M protein mutants were expressed either alone or with Cherry-MYO5B+D in MDCK cells. Cells were immunostained for endogenous Rab10 and GM130. Z axis projections are shown below X-Y slice images, with the merged overlap at the right. Bar=5 μm. Results are representative of 4 individual experiments.

FIGS. 5A and 5B show localization of MHV M-GFP with Cherry-MYO5B+D with Rab8a and the Golgi apparatus. FIG. 5A shows expression of MHV M-GFP alone and with Cherry-MYO5B+D in MDCK cells with immunostaining for endogenous Rab8a. FIG. 5B shows expression of MHV M-GFP alone and with Cherry-MYO5B+D in MDCK cells with immunostaining for endogenous Rab11a and GM130 as a marker of the Golgi apparatus. Z axis projections are shown below X-Y slice images, with the merged overlap at the right. Bar=5 μm. Results are representative of 3 individual experiments.

FIGS. 6A to 6C show colocalization analysis for dual expression of M proteins lo with MYO5B. 3-dimensional Manders' coefficients were calculated for Z-stack images for dually transfected MDCK cells. Dual expression pairs are noted on the X-axis. FIG. 9A shows MHV M-GFP expression with MYO5B+D or MYO5BDD (MYO5B-D). N≥9. *p<0.001 vs MHV M expressed with MYO5B+D. **p<0.0001 vs MHV M expressed with MYO5B+D. FIG. 6B shows M-GFP proteins from PEDV, MERS and SARS-CoV-2 co-expressed with either MYO5B+D or MYO5BDD (MYO5B-D). **p<0.0001 vs M protein expressed with MYO5B+D. N≥9. FIG. 9C shows colocalization of Cherry-MYO5B+D with M-GFP chimeras for PEDV, MERS and SARS-CoV-2 compared with E to K mutants of each M protein. N≥6. *p<0.001 vs between mutant and wild type M protein expressed with MYO5B+D. **p<0.0001 vs between mutant and wild type M protein expressed with MYO5B+D.

FIG. 7 shows immunoprecipitation of MHV M-GFP with Cherry-MYO5B+D. MDCK cells were transfected with MHV M-GFP (M) alone or with Cherry-MYO5B+D (5B). Cell lysates were immunoprecipitated with antibodies against mCherry, resolved on SDS-PAGE, and immunoprecipitates and the original lysates (shown at right) were probed for GFP. Arrowheads at right demonstrate the positions of the doublet for MHV M-GFP in the lysates. Note that only the upper band, representative of the O-glycosylated form of MHV M, was immunoprecipitated with Cherry-MYO5B+D. Molecular weight markers (MW) are indicated at left (kDa). Results are representative of 3 separate experiments.

FIG. 8 shows localization of MHV M-GFP with Cherry-MYO5B+D co-expressed in A549 lung cells. MHV M-GFP was expressed in A549 lung cells alone, with Cherry-MYO5B+D or with Cherry-MYO5BΔD. All cells were immunostained for ERGIC-53 and for F-actin with fluorescent phalloidin. Z axis projections are shown below X-Y slice images, with the merged overlap at the right. Bar=5 μm. Results are representative of 3 individual experiments.

FIGS. 9A to 9C show point mutants in MHV M cytoplasmic tail block co-localization with co-expressed Cherry-MYO5B+D in A549 cells. A549 lung cells were transfected with MHV H-GFP (FIG. 9A), MHV M(E121K)-GFP (FIG. 9B), or MHV M(M126T)-GFP (FIG. 9C) alone, with MYO5B+D or with MYO5BΔD. All cells were co-immunostained for endogenous Rab10 and GM130 as a marker of the Golgi apparatus. Z axis projections are shown below X-Y slice images, with the merged overlap at the right. Bar=5 μm. Results are representative of 3 individual experiments.

FIGS. 10A to 10C show co-localization of coronavirus M proteins with co-expressed MYO5B+D in A549 cells. FIGS. 10A to 10C show PEDV M-GFP (FIG. 10A), MERS M-GFP (FIG. 10B), and SARS-CoV-2 M-GFP (FIG. 10C) expressed in A549 lung cells alone, with Cherry MYO5B+D or Cherry-MYO5BΔD. Z axis projections are shown below X-Y slice images, with the merged overlap at the right. Bar=5 μm. Results are representative of 3 individual experiments.

FIG. 11 shows an assay for protein interaction between MYO5B(ABCDE) and MHV M tail. Yeast harboring pBD-MHV M tail with either pAD-MYO5B(ABCDE) (top line) or pAD-MYO5B(ABCE) (bottom line) were grown overnight (n=6 wells) and then lysed with Y-PER and CPRG was added. The CPRG signal was assayed at 578 nm at room temperature for 200 min in a microtiter plate reader. Data at each time point are shown with standard deviation. Note the linear β-galactosidase activity over the time course.

FIGS. 12A to 12K show A549 lung cells co-expressing MHV M-GFP and Cherry-MYO5B+D tail treated with putative inhibitory small molecules. The human lung cell line A549 was plated onto coverslips and allowed to attach for 24 hours. The cells were then co-transfected with the MHV M-eGFP and mCherry-MYO5B+D(902T) vectors using Polyjet. Twenty-four to 48 hours after transfection the cells were treated with nizatidine at the indicated concentrations or the solvent used to dissolve the drug in, DMSO, for another 24 hours. Cells were fixed in 4% paraformaldehyde and counterstained with phalloidin to visualize the F-actin. Cells were imaged on a Zeiss LSM 710 confocal microscope using a 63×/1.40 Plan-Apochromat oil immersion lens and 2× zoom.

FIGS. 13A to 13F show evaluation of a dose response for inhibition of MHV M-GFP and Cherry-MYO5B+D tail by nizatidine. A549 cells expressing MHV M-GFP and Cherry-MYO5B+D tail were grown for 24 hours in varying concentrations of nizatidine and then stained for F-actin. Nizatidine distrupted co-localization of MHV M-GFP and Cherry-MYO5B+D tail down to 100 μM.

FIGS. 14A to 14C show co-localization of coronavirus M proteins with coexpressed MYO5B+D in A549 cells. PEDV M-GFP (FIG. 14A), MERS M-GFP (FIG. 14B), and SARS-CoV-2 M-GFP (FIG. 14C) were expressed in A549 lung cells alone, with Cherry MYO5B+D or Cherry-MYO5BDD. All cells were co-immunostained for endogenous Rab10 and GM130 as a marker of the Golgi apparatus. Z axis projections are shown below X-Y slice images, with the merged overlap at the right. Bar=5 μm. Results are representative of 3 individual experiments.

FIGS. 15A to 15C show loss of co-localization of coronavirus M proteins with coexpressed MYO5B+D with E to K mutations in A549 cells. E to K mutants of PEDV M-GFP (FIG. 15A), MERS M-GFP (FIG. 15B), and SARS-CoV-2 M-GFP (FIG. 15C) were expressed in A549 lung cells alone or with Cherry MYO5B+D. All cells were co-stained with phalloidin to visualize F-actin. Z axis projections are shown below X-Y slice images, with the merged overlap at the right. Bar=5 μm. Results are representative of 3 individual experiments.

FIGS. 16A to 16C show knockdown of Rab10 expression in A549 cells alters localization of Coronavirus M proteins co-expressed Cherry-MYO5B+D tail. A549 lung cells were transfected with PEDV M-GFP (FIG. 16A), MERS M-GFP (FIG. 16B), and SARS-CoV-2 M-GFP (FIG. 16C) with Cherry-MYO5B+D 902 tail (902T) in non-targeting (NT) wild type cells, Rab10 knockdown (KD) cells and Rab10 knockdown cells with rescue with reexpression of Cer-Rab10. All cells were co-immunostained for endogenous Rab10 and the Golgi marker GM130 except for Cer-Rab10 rescue where Cer-Rab10 fluorescence is shown. Arrowheads in Rab10 knockdown panels show position of residual Rab10 staining. Z axis projections are shown below X-Y slice images, with the merged overlap at the right. Bar=5 μm. Results are representative of 3 individual experiments.

FIGS. 17A to 17D show knockdown of Rab10 expression in A549 cells significantly decreases co-localization of Coronavirus M proteins co-expressed Cherry-MYO5B+D tail. A549 lung cells were transfected with MHV M-GFP, PEDV MGFP, MERS M-GFP or SARS-CoV-2 M-GFP along with Cherry-MYO5B+D 902 tail (902T) in non-targeting (NT) wild type cells, Rab10 knockdown (KD) cells and Rab10 knockdown cells with rescue with re-expression of Cer-Rab10. 3-dimensional Manders' coefficients were calculated for Z-stack images for dually transfected A549 cells. *p<0.01 compared with control NT ShRNA expressing cells. **p<0.001 compared with control NT ShRNA expressing cells.

FIGS. 18A to 18C show colocalization analysis for dual expression of M proteins with MYO5B in A549 cells. 3-dimensional Manders' coefficients were calculated for Z-stack images for dually transfected A549 cells. Dual expression pairs are noted on the X-axis. A. MHV M-GFP expression with MYO5B+D or MYO5BDD (MYO5B−D). N>9. *p<0.001 vs MHV M expressed with MYO5B+D. **p<0.0001 vs MHV M expressed with MYO5B+D. B. M-GFP proteins from PEDV, MERS and SARS-CoV-2 co-expressed with either MYO5B+D or MYO5BDD (MYO5B-D). **p<0.0001 vs M protein expressed with MYO5B+D. N>9. C. Colocalization of Cherry-MYO5B+D with M-GFP chimeras for PEDV, MERS and SARS-CoV-2 compared with E to K mutants of each M protein. N>6. For FIG. 18A, *p<0.001 versus MHV M co-expresssed with MYO5B+D. For FIG. 18B, *p<0.001 between wild type M protein expressed with MYO5B+D vs MYO5B−D. **p<0.0001 between wild type M protein expressed with MYO5B+D vs MYO5B−D. For FIG. 18C, *p<0.001 between mutant and wild type M protein expressed with MYO5B+D. **p<0.0001 between mutant and wild type M protein expressed with MYO5B+D. Analysis was performed on a minimum of 3 fields from at least 3 separate experiments.

FIG. 19 shows knockdown of Rab10 causes loss of colocalization of MHV M protein with MYO5B+D. Non-targeting wild type and Rab10 knockdown (KD) A549 cells were dual transfected with mCherry-MYO5B+D tail (902T; amino acids 902-1848) and MHV M-GFP. Cells were co-immunostained for endogenous Rab10 (cyan) and the Golgi marker GM130 except for Cer-Rab10 rescue where Cer-Rab10 fluorescence is shown. Images are representative of 3 separate experiments. Note that Rab10 KD causes a loss of co-localization of MHV M-GFP with mCherry-MYO5B+D tail.

FIG. 20 shows Nizatidine disrupts the colocalization of MHV M-GFP with ch-MYO5D+D 902 Tail (902T).

FIGS. 21A and 21B show Nizatidine disrupts the colocalization of SARS CoV2 M-GFP with ch-MYO5D+D 902 Tail (902T) in MDCK cells (FIG. 21A) and A549 cells (FIG. 21B).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

“Binding” as used herein (e.g. with reference to a binding domain of a polypeptide) refers to a non-covalent interaction between macromolecules (e.g., between two proteins). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Binding interactions are generally characterized by a dissociation constant (Kd) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower Kd.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰M⁻¹, 10¹¹ M⁻¹, and 10¹² M⁻¹ or more) with that second molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “prevent” refers to a treatment that forestalls or slows the onset of a disease or condition or reduced the severity of the disease or condition. Thus, if a treatment can treat a disease in a subject having symptoms of the disease, it can also prevent that disease in a subject who has yet to suffer some or all of the symptoms.

The term “agent” or “compound” as used herein refers to a chemical entity or biological product, or combination of chemical entities or biological products, administered to a subject to treat or prevent or control a disease or condition. The chemical entity or biological product is preferably, but not necessarily a low molecular weight compound, but may also be a larger compound, or any organic or inorganic molecule, including modified and unmodified nucleic acids such as antisense nucleic acids, RNAi, such as siRNA or shRNA, peptides, peptidomimetics, receptors, ligands, and antibodies, aptamers, polypeptides, nucleic acid analogues or variants thereof. For example, an agent can be an oligomer of nucleic acids, amino acids, or carbohydrates including, but not limited to proteins, peptides, oligonucleotides, ribozymes, DNAzymes, glycoproteins, RNAi agents (e.g., siRNAs), lipoproteins, aptamers, and modifications and combinations thereof. In some embodiments, an active agent is a nucleic acid, e.g., miRNA or a derivative or variant thereof.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 50, 60, 70, 80, 90, 100%, or any amount of reduction in between as compared to native or control levels.

The term “antibody” refers to natural or synthetic antibodies that selectively bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that selectively bind the target antigen.

The term “small molecule” refers to a molecule, such as an organic or organometallic compound, with a molecular weight of less than 2,000 Daltons, more preferably less than 1,500 Daltons, most preferably less than 1,000 Daltons. The small molecule can be a hydrophilic, hydrophobic, or amphiphilic compound.

The term “variant” refers to an amino acid sequence having conservative amino acid substitutions, non-conservative amino acid substitutions (i.e. a degenerate variant), or a peptide having 60%, 65%, 70%, 71%, 72%, 73%, 74%, 75%, 76%, 77%, 78%, 79%, 80%, 81%, 82%$, 83%, 84%, 85%, 86%, 87%, 88%, 89%, 90%, 91%, 92%, 93%, 94%, 95%, 96%, 97%, 98%, or 99% sequence identity to the recited sequence.

The term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods. For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

### Coronavirus

Coronaviruses are a group of related RNA viruses that cause diseases in mammals and birds. In humans and birds, they cause respiratory tract infections that can range from mild to lethal. Coronaviruses constitute the subfamily Orthocoronavirinae, in the family Coronaviridae, order Nidovirales, and realm Riboviria. Coronaviruses are divided into the four genera: Alphacoronavirus, Betacoronavirus, Gammacoronavirus and Deltacoronavirus. Alphacoronaviruses and betacoronaviruses infect mammals, while gammacoronaviruses and deltacoronaviruses primarily infect birds. Therefore, in some embodiments, the coronavirus is an alphacoronavirus or betacoronavirus.

Alphacoronaviruses (Alpha-CoV) that infect humans include Human coronavirus 229E (HCoV-229E) and Human coronavirus NL63 (HCoV-N L63).

Betacoronaviruses (BetaCoVs) is composed of four varying viral lineages: A, B, C, D. The betacoronaviruses of the greatest clinical importance concerning humans are OC43 and HKU1 of lineage A, SARS-CoV and SARS-CoV-2 (which causes the disease COVID-19) of lineage B, and MERS-CoV of lineage C. In some embodiments, the betacoronavirus is a lineage A, B, C, or D betacoronavirus. In some embodiments, the disclosed compositions and methods can be used to treat any betacoronavirus. In some embodiments, the disclosed compositions and methods can be used to treat a betacoronavirus of genus A, B, C, or D. Therefore, in some embodiments, the disclosed compositions and methods can be used to treat SARS-CoV-2.

### Interruption of the Interaction Between Coronavirus M Proteins and MYO5B+D

### Dominant Negative Polypeptides

In some embodiments, the interaction between M protein and MYO5B is disrupts with a dominant negative polypeptide that competes for binding of coronavirus M protein to endogenous MYO5B.

For example, in some embodiments, the dominant negative polypeptide is a soluble M protein capable of binding and sequestering MYO5B, thereby preventing M protein on coronavirus from binding MYO5B. Soluble receptors include can be produced by fusing a fragment of M protein containing the binding site for MYO5B, e.g. lacking the transmembrane domain, to other functional and structural proteins including, but not limited to, a human Fc (e.g. human Fc derived from IgG1, IgG2, IgG3, IgG4, IgA1, IgA2, IgD, IgE, IgM); protein tags (e.g. myc, FLAG, GST). In certain embodiments a fragment of M protein is linked to human IgG1 Fc.

In some embodiments, MHV M protein has the amino acid sequence:

(SEQ ID NO: 1)
MSSTTQAPEP VYQWTADEAV QFLKEWNFSL GIILLFITII LQFGYTSRSM
FIYVVKMIIL WLMWPLTIVL CIFNCVYALN NVYLGFSIVF TIVSIVIWIM YFVNSIRLFI
RTGSWWSFNP ETNNLMCIDM KGTVYVRPII EDYHTLTATI IRGHLYMQGV
KLGTGFSLSD LPAYVTVAKV SHLCTYKRAF LDKVDGVSGF AVYVKSKVGN
YRLPSNKPSGA DTALLRI.

In some embodiments, the C-terminal tail binding region of MHV M includes amino acids 105-208 of SEQ ID NO:1. Therefore, in some embodiments, the soluble M protein has the amino acid sequence:

(SEQ ID NO: 2)
SIRLFI RTGSWWSFNP ETNNLMCIDM KGTVYVRPII EDYHTLTATI
IRGHLYMQGV KLGTGFSLSD LPAYVTVAKV SHLCTYKRAF
LDKVDGVSGF AVYVKSKV.

In some embodiments MFRS M nrotein has the amino acid seauence:

(SEQ ID NO: 3)
MSNMTQLTEA QIIAIIKDWN FAWSLIFLLI TIVLQYGYPS
RSMTVYVFKM FVLWLLWPSS MALSIFSAVY PIDLASQIIS
GTVAAVSAMM WISYFVQSIR LFMRTGSWWS FNPETNCLLN
VPFGGTTVVR PLVEDSTSVT AVVTNGHLKM AGMHFGACDY
DRLPNEVTVA KPNVLIALKM VKRQSYGTNS GVAIYHRYKA
GNYRSPPITA DIELALLRA.

In some embodiments, the C-terminal tail binding region of MERS M includes amino acids 98-219 of SEQ ID NO:3. Therefore, in some embodiments, the soluble M protein has the amino acid sequence:

(SEQ ID NO: 4)
SIR LFMRTGSWWS FNPETNCLLN VPFGGTTVVR PLVEDSTSVT
AVVTNGHLKM AGMHFGACDY DRLPNEVTVA KPNVLIALKM
VKRQSYGTNS GVAIYHRYKA GNYRSPPITA DIELALLRA.

In some embodiments, SARS M protein has the amino acid sequence:

(SEQ ID NO: 5)
MAENDTITVD QLKHLLEQWN LVIGFLFFAW ILLLQFAYSN
RNRFLYIIKL VFLWLLWPIT LACFVLAAVY RINWATGGIA
IAMACLVGLM WLSYFVASFR LFARTRSWWS FNPETNILLN
VPLRGSIITR PLLESELVIG AVIIRGYLRM AGHSLGRCDI
KDLPKEITVA TSRTLSYYRL GASQRVGTDS GFAVYHRYRI
GNYKLNTDHS GSNDNIALLV Q.

In some embodiments, the C-terminal tail binding region of SARS M includes amino acids 98-221 of SEQ ID NO:5. Therefore, in some embodiments, the soluble M protein has the amino acid sequence:

(SEQ ID NO: 6)
SFR LFARTRSWWS FNPETNILLN VPLRGSIITR PLLESELVIG
AVIIRGYLRM AGHSLGRCDI KDLPKEITVA TSRTLSYYRL
GASQRVGTDS GFAVYHRYRI GNYKLNTDHS GSNDNIALLV Q.

In some embodiments, SARS-CoV-2 M protein has the amino acid sequence:

(SEQ ID NO: 7)
MADSNGTITV EELKKLLEQW NLVIGFLFLT WICLLQFAYA
NRNRFLYIIK LIFLWLVWPV TLACFVLAAV YRINWITGGI
AIAMACLVRL MWLSYFIASF RLFARTRSMW SFNPETNILL
NVPLHGTILT RPLLESELVI GAVILRGHLR IAGHHLGRCD
IKDLPKEITV ATSRTLSYYK LGASQRVAGD SGFAAYSRYR
IGNYKLNTDH SSSSDNIALL VQ.

In some embodiments, the C-terminal tail binding region of SARS-CoV-2 M includes amino acids 99-222 of SEQ ID NO:8. Therefore, in some embodiments, the soluble M protein has the amino acid sequence:

(SEQ ID NO: 8)
F RLFARTRSMW SFNPETNILL NVPLHGTILT RPLLESELVI
GAVILRGHLR IAGHHLGRCD IKDLPKEITV ATSRTLSYYK
LGASQRVAGD SGFAAYSRYR IGNYKLNTDH SSSSDNIALL VQ.

In some embodiments, the dominant negative polypeptide is a MYO5B fragment capable of competing with endogenous MYO5B for binding to betacoronavirus M protein.

In some embodiments, human MYO5B+D has the amino acid sequence:

(SEQ ID NO: 9)
MSVGELYSQC TRVWIPDPDE VWRSAELTKD YKEGDKSLQL
RLEDETILEY PIDVQRNQLP FLRNPDILVG ENDLTALSYL
HEPAVLHNLK VRFLESNHIY TYCGIVLVAI NPYEQLPIYG
QDVIYTYSGQ NMGDMDPHIF AVAEEAYKQM ARDEKNQSII
VSGESGAGKT VSAKYAMRYF ATVGGSASET NIEEKVLASS
PIMEAIGNAK TTRNDNSSRF GKYIQIGFDK RYHIIGANMR
TYLLEKSRVV FQADDERNYH IFYQLCAAAG LPEFKELALT
SAEDFFYTSQ GGDTSIEGVD DAEDFEKTRQ AFTLLGVKES
HQMSIFKIIA SILHLGSVAI QAERDGDSCS ISPQDVYLSN
FCRLLGVEHS QMEHWLCHRK LVTTSETYVK TMSLQQVINA
RNALAKHIYA QLFGWIVEHI NKALHTSLKQ HSFIGVLDIY
GFETFEVNSF EQFCINYANE KLQQQFNSHV FKLEQEEYMK
EQIPWTLIDF YDNQPCIDLI EAKLGILDLL DEECKVPKGT
DQNWAQKLYD RHSSSQHFQK PRMSNTAFII VHFADKVEYL
SDGFLEKNRD TVYEEQINIL KASKFPLVAD LFHDDKDPVP
ATTPGKGSSS KISVRSARPP MKVSNKEHKK TVGHQFRTSL
HLLMETLNAT TPHYVRCIKP NDEKLPFHFD PKRAVQQLRA
CGVLETIRIS AAGYPSRWAY HDFFNRYRVL VKKRELANTD
KKAICRSVLE NLIKDPDKFQ FGRTKIFFRA GQVAYLEKLR
ADKFRTATIM IQKTVRGWLQ KVKYHRLKGA TLTLQRYCRG
HLARRLAEHL RRIRAAVVLQ KHYRMQRARQ AYQRVRRAAV
VIQAFTRAMF VRRTYRQVLM EHKATTIQKH VRGWMARRHF
QRLRDAAIVI QCAFRMLKAR RELKALRIEA RSAEHLKRLN
VGMENKVVQL QRKIDEQNKE FKTLSEQLSV TTSTYTMEVE
RLKKELVHYQ QSPGEDTSLR LQEEVESLRT ELQRAHSERK
ILEDAHSREK DELRKRVADL EQENALLKDE KEQLNNQILC
QSKDEFAQNS VKENLMKKEL EEERSRYQNL VKEYSQLEQR
YDNLRDEMTI IKQTPGHRRN PSNQSSLESD SNYPSISTSE
IGDTEDALQQ VEEIGLEKAA MDMTVFLKLQ KRVRELEQER
KKLQVQLEKR EQQDSKKVQA EPPQTDIDLD PNADLAYNSL
KRQELESENK KLKNDLNELR KAVADQATQN NSSHGSPDSY
SLLLNQLKLA HEELEVRKEE VLILRTQIVS ADQRRLAGRN
AEPNINARSS WPNSEKHVDQ EDAIEAYHGV CQTNSKTEDW
GYLNEDGELG LAYQGLKQVA RLLEAQLQAQ SLEHEEEVEH
LKAQLEALKE EMDKQQQTFC QTLLLSPEAQ VEFGVQQEIS
RLTNENLDLK ELVEKLEKNE RKLKKQLKIY MKKAQDLEAA
QALAQSERKR HELNRQVTVQ RKEKDFQGML EYHKEDEALL
IRNLVTDLKP QMLSGTVPCL PAYILYMCIR HADYTNDDLK
VHSLLTSTIN GIKKVLKKHN DDFEMTSFWL SNTCRLLHCL
KQYSGDEGFM TQNTAKQNEH CLKNFDLTEY RQVLSDLSIQ
IYQQLIKIAE GVLQPMIVSA MLENESIQGL SGVKPTGYRK
RSSSMADGDN SYCLEAIIRQ MNAFHTVMCD QGLDPEIILQ
VFKQLFYMIN AVTLNNLLLR KDVCSWSTGM QLRYNISQLE
EWLRGRNLHQ SGAVQTMEPL IQAAQLLQLK KKTQEDAEAI
CSLCTSLSTQ QIVKILNLYT PLNEFEERVT VAFIRTIQAQ
LQERNDPQQL LLDAKHMFPV LFPFNPSSLT MDSIHIPACL
NLEFLNEV.

In some embodiments, exons ABODE of MYO5B (1235-1439) are involved in binding to M protein. Therefore, in some embodiments, the MYO5B fragment comprises the amino acid sequence:

(SEQ ID NO: 10)
GSPDSY SLLLNQLKLA HEELEVRKEE VLILRTQIVS ADQRRLAGRN
AEPNINARSS WPNSEKHVDQ EDAIEAYHGV CQTNSKTEDW
GYLNEDGELG LAYQGLKQVA RLLEAQLQAQ SLEHEEEVEH
LKAQLEALKE EMDKQQQTFC QTLLLSPEAQ VEFGVQQEI
RLTNENLDLK ELVEKLEKNE RKLKKQLKIY MKKAQDLEA

In some embodiments, exon D of MYO5B (1315-1340) is required for association with MHV M. Therefore, in some embodiments, the MYO5B fragment comprises the amino acid sequence: SKTEDWGYLNEDGELGLAYQGLKQVA (SEQ ID NO:11).

### Antibodies and Aptamers

In some embodiments, the interaction between M protein and MYO5B is disrupts with an antibody or aptamer that selectively binds M protein or MYO5B. The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof.

Antibodies that can be used in the disclosed compositions and methods include whole immunoglobulin (i.e., an intact antibody) of any class, fragments thereof, and synthetic proteins containing at least the antigen binding variable domain of an antibody. The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies.

Also disclosed are fragments of antibodies which have bioactivity. The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment.

Techniques can also be adapted for the production of single-chain antibodies specific to an antigenic protein of the present disclosure. Methods for the production of single-chain antibodies are well known to those of skill in the art. A single chain antibody can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation.

Divalent single-chain variable fragments (di-scFvs) can be engineered by linking two scFvs. This can be done by producing a single peptide chain with two VH and two VL regions, yielding tandem scFvs. ScFvs can also be designed with linker peptides that are too short for the two variable regions to fold together (about five amino acids), forcing scFvs to dimerize. This type is known as diabodies. Diabodies have been shown to have dissociation constants up to 40-fold lower than corresponding scFvs, meaning that they have a much higher affinity to their target. Still shorter linkers (one or two amino acids) lead to the formation of trimers (triabodies or tribodies). Tetrabodies have also been produced. They exhibit an even higher affinity to their targets than diabodies.

The term “aptamer” refers to oligonucleic acid or peptide molecules that bind to a specific target molecule. These molecules are generally selected from a random sequence pool. The selected aptamers are capable of adapting unique tertiary structures and recognizing target molecules with high affinity and specificity. A “nucleic acid aptamer” is a DNA or RNA oligonucleic acid that binds to a target molecule via its conformation, and thereby inhibits or suppresses functions of such molecule. A nucleic acid aptamer may be constituted by DNA, RNA, or a combination thereof. A “peptide aptamer” is a combinatorial protein molecule with a variable peptide sequence inserted within a constant scaffold protein. Identification of peptide aptamers is typically performed under stringent yeast dihybrid conditions, which enhances the probability for the selected peptide aptamers to be stably expressed and correctly folded in an intracellular context.

Peptide aptamer selection can be made using different systems, but the most used is currently the yeast two-hybrid system. Peptide aptamer can also be selected from combinatorial peptide libraries constructed by phage display and other surface display technologies such as mRNA display, ribosome display, bacterial display and yeast display. These experimental procedures are also known as biopannings. Among peptides obtained from biopannings, mimotopes can be considered as a kind of peptide aptamers. All the peptides panned from combinatorial peptide libraries have been stored in a special database with the name MimoDB.

### Small Molecules

In some embodiments, the interaction between M protein and MYO5B is disrupts with a small molecule, e.g. identified by the methods described below.

It is understood that some small molecules may have been described for use in treating coronavirus by a different mechanism that could also disrupt the interaction between M protein and MYO5B. For example, Ishola, A A, et al. J Biomol Struct Dyn 2021 1-18 suggest the use of histamine H2-receptor antagonists, such as Cimetidine, Famotidine, Nizatidine, Ranitidine, for the treatment of COVID. Amiodorone, Nimesulide, and Diclofenac have also been suggested for use in treating COVID by a different mechanism. To the extent that any of these small molecules also disrupt the interaction between M protein and MYO5B, these small molecules are in some embodiments, excluded from the disclosed methods.

### Screening Method

Also provided herein is a method of identifying an agent that can be used to treat a coronavirus. The method can comprise providing a sample comprising coronavirus membrane glycoprotein (M protein) and human Myosin Vb protein (MYO5B) under conditions that allow the binding of M protein and MYO5B, contacting the sample with a candidate agent, detecting the level of M protein/MYO5B binding, comparing the binding level to a control, a decrease in M protein/MYO5B binding compared to the control identifying an agent that can be used to treat a betacoronavirus.

The binding of M protein to MYO5B can be detected using routine methods, such as immunodetection methods, that do not disturb protein binding. The methods can be cell-based or cell-free assays. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In some embodiments, the method involves use of an interaction trap assay, also known as the “two hybrid assay,” for identifying agents that disrupt or potentiate interaction between M protein and MYO5B. See for example, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696). In some embodiments, the method involves use of a reverse two hybrid system to identify compounds (e.g., small molecules or peptides) that dissociate interactions between M protein and MYO5B. See for example, Vidal and Legrain, (1999) Nucleic Acids Res 27:919-29; Vidal and Legrain, (1999) Trends Biotechnol 17:374-81; and U.S. Pat. Nos. 5,525,490; 5,955,280; and 5,965,368.

The premise behind the two-hybrid assay is the activation of downstream reporter gene(s) by the binding of a transcription factor onto an upstream activating sequence (UAS). For two-hybrid screening, the transcription factor is split into two separate fragments, called the DNA-binding domain (DBD or often also abbreviated as BD) and activating domain (AD). The BD is the domain responsible for binding to the UAS and the AD is the domain responsible for the activation of transcription.

The most common screening approach is the yeast two-hybrid assay. This system often utilizes a genetically engineered strain of yeast in which the biosynthesis of certain nutrients (usually amino acids or nucleic acids) is lacking. When grown on media that lacks these nutrients, the yeast fail to survive. Plasmids are engineered to produce a protein product in which the DNA-binding domain (BD) fragment is fused onto a protein while another plasmid is engineered to produce a protein product in which the activation domain (AD) fragment is fused onto another protein. The protein fused to the BD may be referred to as the bait protein. If the bait and prey proteins interact (i.e., bind), then the AD and BD of the transcription factor are indirectly connected, bringing the AD in proximity to the transcription start site and transcription of reporter gene(s) can occur. If the two proteins do not interact, there is no transcription of the reporter gene. In this way, a successful interaction between the fused protein is linked to a change in the cell phenotype.

In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) used.

Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available, e.g., from purveyors of chemical libraries including but not limited to ChemBridge Corporation (16981 Via Tazon, Suite G, San Diego, CA, 92127, USA, www.chembridge.com); ChemDiv (6605 Nancy Ridge Drive, San Diego, CA 92121, USA); Life Chemicals (1103 Orange Center Road, Orange, CT 06477); Maybridge (Trevillett, Tintagel, Cornwall PL34 OHW, UK)

Alternatively, libraries of natural compounds in the form of bacterial, fungal, plant, and animal extracts are commercially available from a number of sources, including O2H, (Cambridge, UK), MerLion Pharmaceuticals Pte Ltd (Singapore Science Park II, Singapore 117528) and Galapagos NV (Generaal De Wittelaan L11 A3, B-2800 Mechelen, Belgium).

In addition, natural and synthetically produced libraries are produced, if desired, according to methods known in the art, e.g., by standard extraction and fractionation methods or by standard synthetic methods in combination with solid phase organic synthesis, micro-wave synthesis and other rapid throughput methods known in the art to be amenable to making large numbers of compounds for screening purposes. Furthermore, if desired, any library or compound, including sample format and dissolution is readily modified and adjusted using standard chemical, physical, or biochemical methods. In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their effect should be employed whenever possible.

When a crude extract is found to have a desired activity, further fractionation of the positive lead extract is necessary to isolate chemical constituents responsible for the observed effect. The same assays described herein for the detection of activities in mixtures of compounds can be used to purify the active component and to test derivatives thereof. Methods of fractionation and purification of such heterogeneous extracts are known in the art. If desired, compounds shown to be useful agents for treatment are chemically modified according to methods known in the art. Compounds identified as being of therapeutic value may be subsequently analyzed using in vitro cell based models and animal models for diseases or conditions, such as those disclosed herein.

Candidate agents encompass numerous chemical classes, but are most often organic molecules, e.g., small organic compounds having a molecular weight of more than 100 and less than about 2,500 Daltons. Candidate agents can include functional groups necessary for structural interaction with proteins, particularly hydrogen bonding, and typically include at least an amine, carbonyl, hydroxyl or carboxyl group, for example, at least two of the functional chemical groups. The candidate agents often contain cyclical carbon or heterocyclic structures and/or aromatic or polyaromatic structures substituted with one or more of the above functional groups.

In some embodiments, the candidate agents are proteins. In some aspects, the candidate agents are naturally occurring proteins or fragments of naturally occurring proteins. Thus, for example, cellular extracts containing proteins, or random or directed digests of proteinaceous cellular extracts, can be used. In this way libraries of procaryotic and eucaryotic proteins can be made for screening using the methods herein. The libraries can be bacterial, fungal, viral, and vertebrate proteins, and human proteins.

### Pharmaceutical Compositions

The disclosed therapeutic agents that inhibit the interaction between M protein and MYO5B can be used therapeutically in combination with a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The materials may be in solution, suspension (for example, incorporated into microparticles, liposomes, or cells). Suitable carriers and their formulations are described in Remington: The Science and Practice of Pharmacy (22nd ed.) eds. Loyd V. Allen, Jr., et al., Pharmaceutical Press, 2012. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of polypeptides to humans, including solutions such as sterile water, saline, and buffered solutions at physiological pH. Pharmaceutical compositions may include carriers, thickeners, diluents, buffers, preservatives, surface active agents and the like in addition to the disclosed agents. Pharmaceutical compositions may also include one or more active ingredients such as antimicrobial agents, anti-inflammatory agents, anesthetics, and the like.

The disclosed agents are preferably formulated for delivery via intranasal, intramuscular, subcutaneous, transdermal or sublingual administration. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Some of the compositions may potentially be administered as a pharmaceutically acceptable acid—or base—addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The disclosed agents can further include (or be administered in combination with) one or more of classes of antibiotics, steroids, analgesics, anti-inflammatory agents, anti-histaminic agents, or any combination thereof. Antibiotics include Aminoglycosides, Cephalosporins, Chloramphenicol, Clindamycin, Erythromycins, Fluoroquinolones, Macrolides, Azolides, Metronidazole, Penicillins, Tetracyclines, Trimethoprim-sulfamethoxazole, and Vancomycin. Suitable steroids include andranes, such as testosterone. Narcotic and non-narcotic analgesics include morphine, codeine, heroin, hydromorphone, levorphanol, meperidine, methadone, oxydone, propoxyphene, fentanyl, methadone, naloxone, buprenorphine, butorphanol, nalbuphine, and pentazocine. Anti-inflammatory agents include alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, momiflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium. Anti-histaminic agents include ethanolamines (e.g., diphenhydrmine carbinoxamine), Ethylenediamine (e.g., tripelennamine pyrilamine), Alkylamine (e.g., chlorpheniramine, dexchlorpheniramine, brompheniramine, triprolidine), other anti-histamines like astemizole, loratadine, fexofenadine, bropheniramine, clemastine, acetaminophen, pseudoephedrine, triprolidine).

### Methods of Administration

As disclosed herein can be administering to a subject in need thereof using routine methods. For example, the disclosed agents can be administered intramuscularly, intranasally, or by microneedle in the skin. The compositions may be administered orally, intravenously, subcutaneously, transdermally (e.g., by microneedle), intraperitoneally, ophthalmically, vaginally, rectally, sublingually, or by inhalation.

Parenteral administration of the composition, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. For example, effective dosages and schedules for administering the compositions may be determined empirically, and making such determinations is within the skill in the art. The dosage ranges for the administration of the compositions are those large enough to produce the desired effect in which the symptoms disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. A typical dosage of the disclosed agents used alone might range from about 1 μg/kg to up to 100 mg/kg of body weight or more per vaccination, such as 10 μg/kg to 50 mg/kg, or 50 μg/kg to 10 mg/kg, depending on the factors mentioned above. In addition to dosing by the ratio of mass-of-agent to mass-of-patient, standardized doses for demarcated demographics can also be used. A typical dose for an adult patient may be 1 μg to 1000 μg, or 10 μg to 150 μg, or 15 μg to 135 μg per subject. A typical dose for a child patient may be 1 μg to 1000 μg, or 10 μg to 150 μg, or 15 μg to 135 μg per subject. A typical dose for an elderly patient may be 1 μg to 1000 μg, or 10 μg to 150 μg, or 15 μg to 135 μg per subject.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

The beta-coronavirus family includes multiple pathogenic viruses including Mouse Hepatitis Virus (MHV), MERS, SARS and SARS-CoV-2 (COVI D-19) as well as the common cold virus (Cui, J., et al. Nature reviews. Microbiology 2019 17:181-192). Beta-coronaviruses are assembled from a series of proteins transcribed from the viral genome (Cui, J., et al. Nature reviews. Microbiology 2019 17:181-192). Among these proteins, the membrane glycoprotein, M protein, is the major capsid membrane protein and serves as the nidus for virion protein assembly (Rottier, P J., et al. Adv Exp Med Biol 1990 276:127-135; de Haan, CA., et al. Virology 2003 312:395-406). While trafficking through the Golgi apparatus is clearly necessary for M protein function, previous studies have suggested that virion particles are assembled in the endoplasmic reticulum-Golgi intermediate compartment (ERGIC) (Locker, J K. et al. J Biol Chem 1994 269:28263-28269; Klumperman, J. et al. J Virol 1994 68:6523-6534). Although these studies have presented an order of virion assembly, few investigations have addressed interactions of virion proteins with components of the vesicle trafficking systems in host cells. The association of M protein with host regulators of membrane trafficking was therefore investigated.

### Results

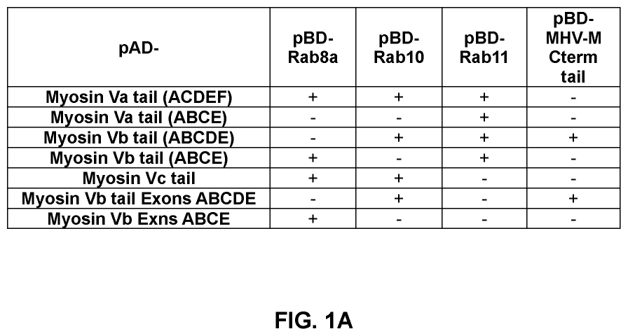

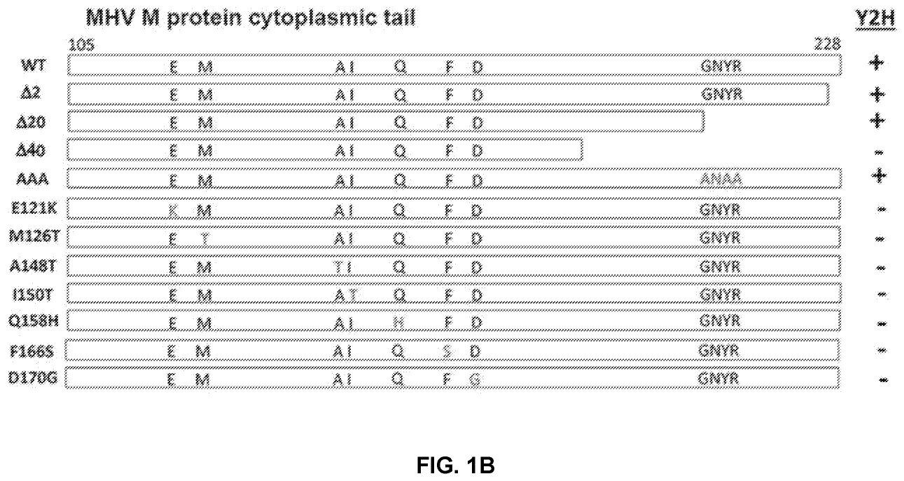

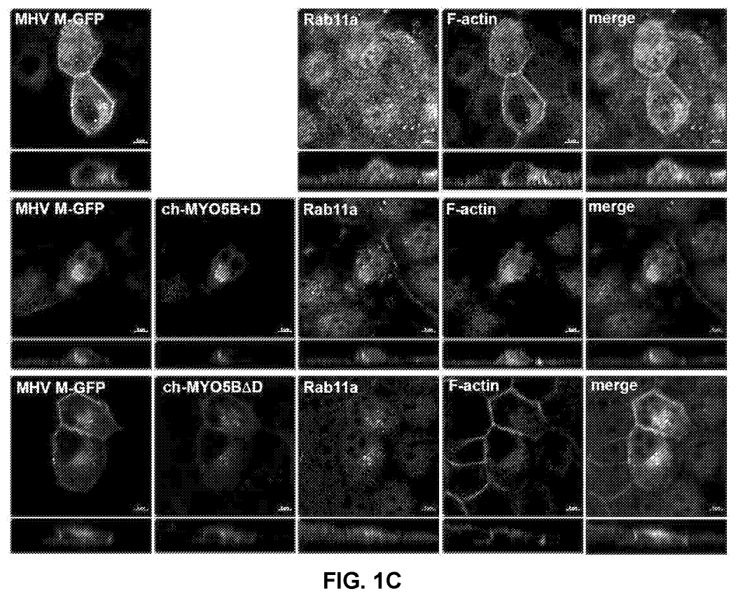

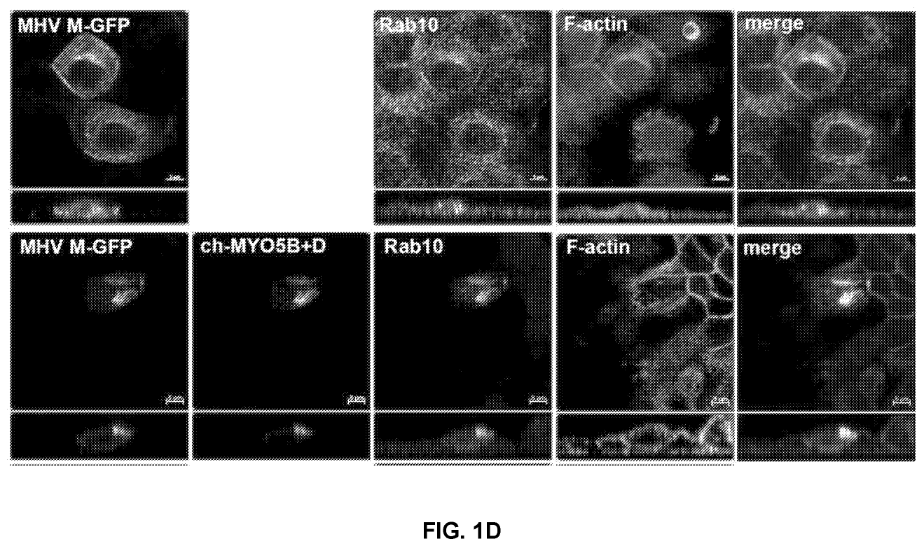

A yeast 2-hybrid screen was performed with the cytoplasmic tail of MHV M protein as a bait to identify interacting proteins in a mouse brain prey library. An interaction of the MHV M protein tail and Myosin Vb (MYO5B) was identified. Sixty clones of Myo5b beginning with amino acids 997 to 1249 and extending to the carboxyl terminal end of the protein were identified. Using a series of 2-hybrid assays, the association was subsequently confirmed, demonstrating that the M cytoplasmic tail interacted with one specific alternatively spliced exon of Myo5b: Exon D (FIG. 1A). Rab10 interacts with the alternatively spliced Exon D in both MYO5A and MYO5B and in a homologous unspliced region in MYO5C (Roland, J T., et al. J Biol Chem 2009 284:1213-1223). Interestingly, while the splice variant of MYO5B without Exon D was ubiquitous, the splice variant with Exon D was detected in the lung, brain, heart and intestines (Roland, J T., et al. J Biol Chem 2009 284:1213-1223). A series of yeast 2-hybrid studies were performed that demonstrated MHV M protein did not interact with either Myosin Va or Myosin Vc sequences, even though they did contain appropriate D exons (FIG. 1A). Furthermore, MHV M tail did not interact with MYO5B sequences without exon D (FIG. 1A). A construct containing just the ABCDE exons also interacted with MHV M, but the ABCE exon sequence did not (FIG. 1A). All of these finding led to the conclusion that M protein of MHV forms a quite specific association with Exon D of MYO5B.

The yeast 2-hybrid assay between MHV M tail and MYO5B ABCDE exons (MYO5B(ABCDE)) was next utilized to evaluate whether truncations of the carboxyl terminus would alter the interaction (FIG. 1B). Previous studies had suggested that deletion of the terminal 2 amino acids of the M tail would impair MHV assembly (de Haan, CA., et al. J Virol 1998 72:6838-6850). However, this truncation had no effect on MYO5B(ABCDE) interaction with MHV M tail. Similarly, truncation of the last 20 amino acids also did not alter interaction (FIG. 1B). This truncation eliminated the conserved region that has been reported as important for Golgi trafficking of MERS M protein (Perrier, A. et al. J Biol Chem 2019 294:14406-14421). Insertion of a triple alanine substitution in this sequence also failed to affect MHV M tail interaction with MYO5B(ABCDE). However, truncation of 40 carboxyl-terminal amino acids abrogated the interaction (FIG. 1B).

In order to provide greater detail on the requirements for interaction, a random mutagenesis of the MHV cytosolic tail was performed to identify mutations that disrupt the interaction with MYO5B(ABCDE). This random mutagenesis was performed with PCR amplification of the ABCDE exons with Taq in the presence of manganese ion. The resulting PCR products were ligated back into pAD to create a library of mutant sequences. This ABCDE library was then transfected into yeast with pBD-MHV M tail, identifying white colonies in the presence of Xgal as putative revertant mutants. FIG. 1B shows the results of those studies, where 7 point mutants were identified that could disrupt the interaction between MYO5B(ABCDE) and MHV M cytoplasmic tail. Interestingly one of the point mutations, E121K, has been evaluated previously in studies looking at the role of an amphipathic sequence that is conserved across all beta-coronavirus M proteins (Arndt, A L., et al. J Virol 2010 84:11418-11428). Those studies demonstrated that the E121K mutation in M blocked assembly of both VLPs and full MHV virus (Arndt, A L., et al. J Virol 2010 84:11418-11428). Thus, at least one of the mutations that can block M interaction with MYO5B+D alters efficient virus assembly.

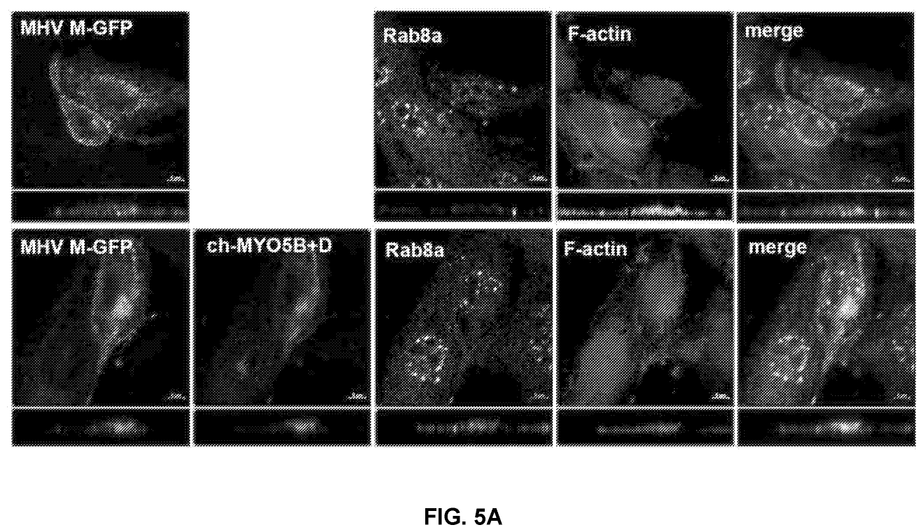

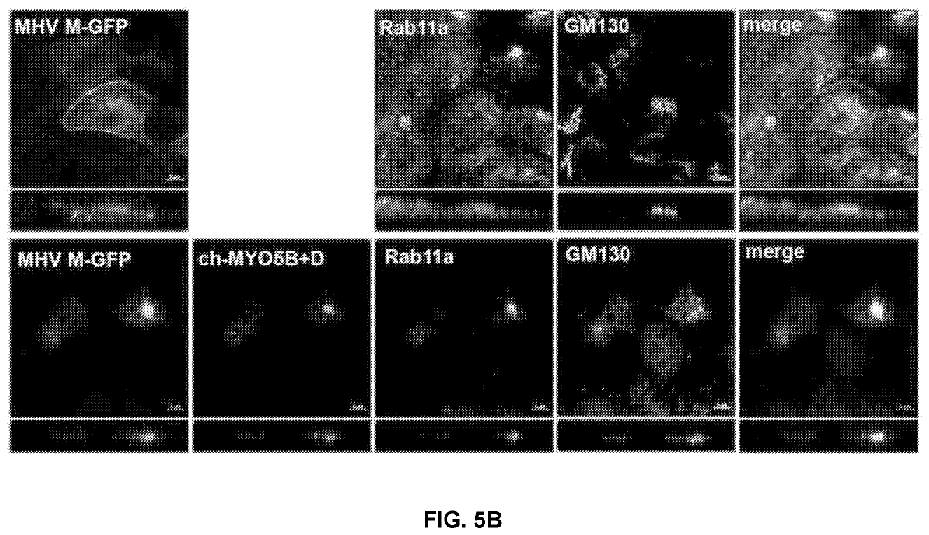

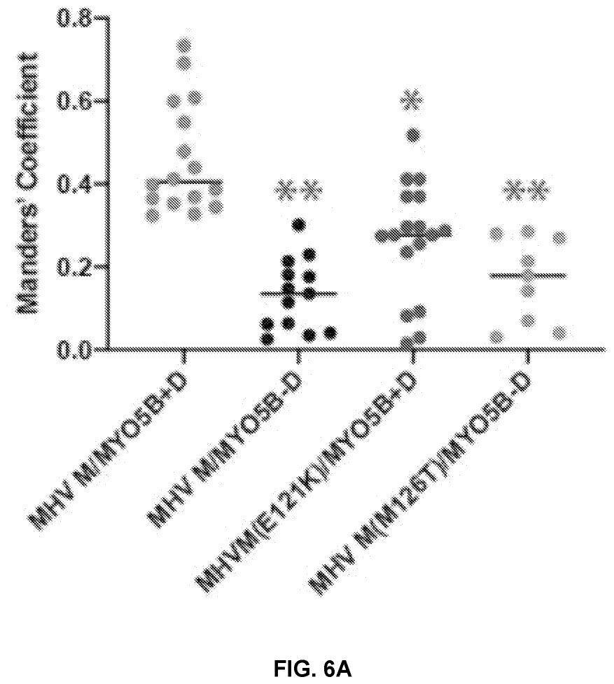

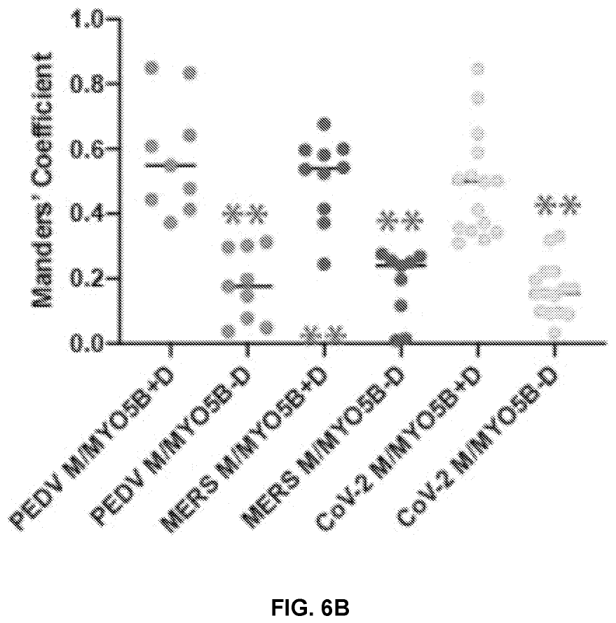

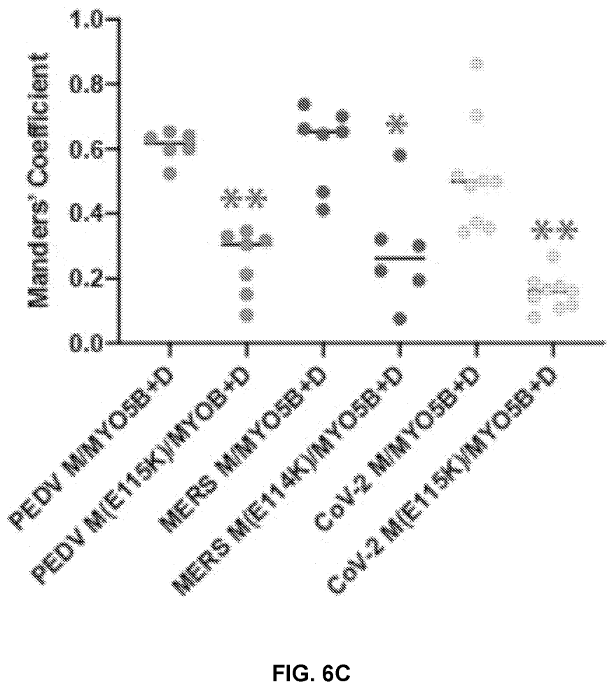

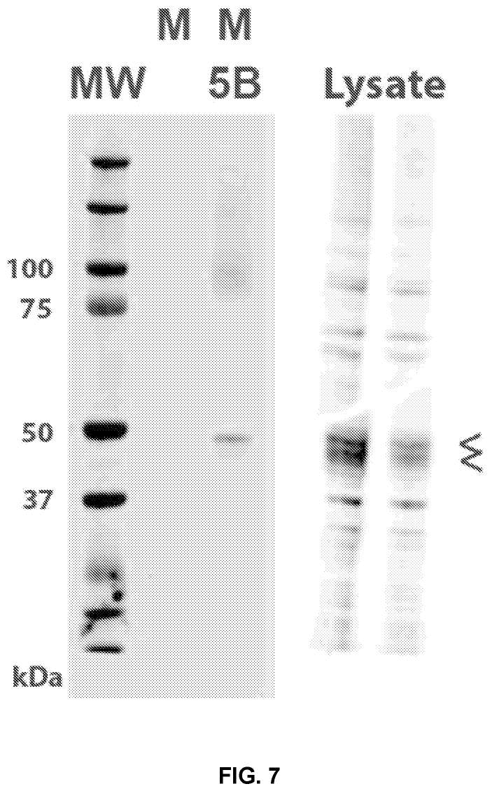

The interaction of MYOS5B+D with MHV M protein following co-transfection into MDCK cells, a polarizing epithelial cell line, was next examined. Vectors coding for MHV M-GFP and Cherry-MYO5B+D full length protein were co-transfected into MDCK cells. FIG. 1C demonstrates that MHV M and MYO5B+D colocalized in membrane vesicles that also contained Rab11a and Rab10. Cherry-MYO5B+D and MHV M-GFP did not colocalize with Rab8a (FIG. 5A). The membranes harboring MHV M-GFP and Cherry-MYO5B+D were distinct from the Golgi membranes (FIG. 5B) and also were consistently associated with an accumulation of F-actin (FIG. 1C). Importantly, when Cherry-MYO5B without the D exon (MYO5BΔD) was co-expressed with MHV M-GFP, little co-localization was observed, and more of the M protein was seen along the lateral and junctional membranes (FIG. 1C). Analysis of Manders' coefficients demonstrated a significant loss in co-localization (FIG. 6A). To confirm the direct interaction of MHV M co-expressed with MYO5B+D, putative complexes were immunoprecipitated with antibodies against mCherry. FIG. 7 demonstrates that MHV M-GFP precipitated with Cherry-MYOB+D. Of interest, however, the co-precipitation of only the higher molecular weight form of MHV M was consistently observed, which represents a Golgi-modified O-glycosylated form (Krijnse-Locker, J., et al. J Cell Biol 1994 124:55-70; de Haan, C A. et al. J Biol Chem 1998 273:29905-29914). These studies suggest that MYO5B+D only interacts with MHV M in Golgi-derived or post-Golgi membranes.

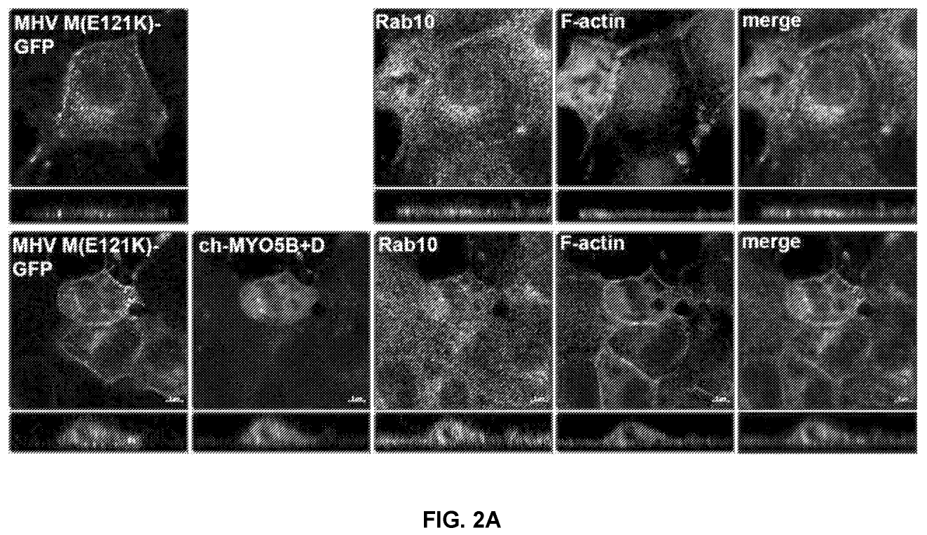

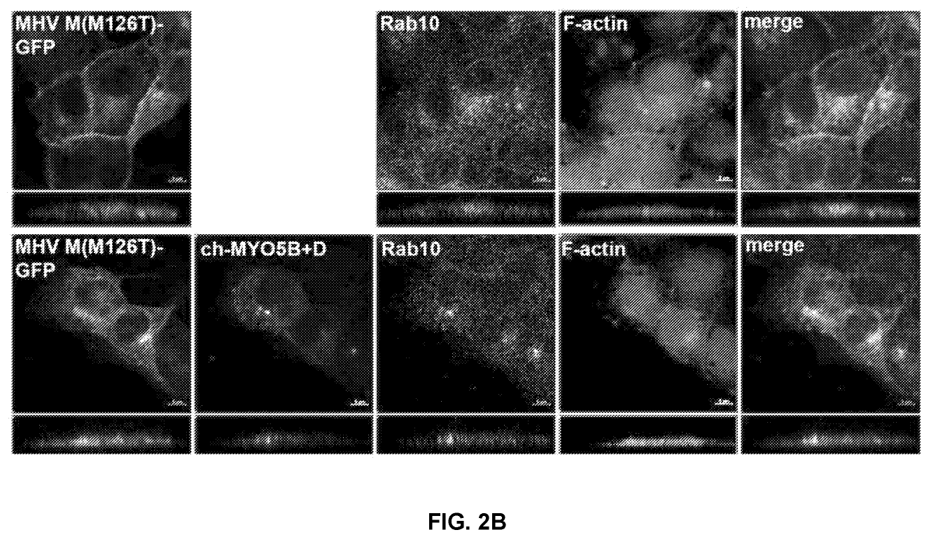

Next evaluated was the effects of the mutations in MHV M identified in the yeast 2-hybrid screen. FIG. 2 demonstrates that both the E121K and the M126T point mutation caused a loss of co-localization with MYO5B+D (FIG. 6A) and Rab10. These findings suggest that point mutations that alter MHV M interactions with MYO5B canaffect trafficking of the viral M glycoprotein.

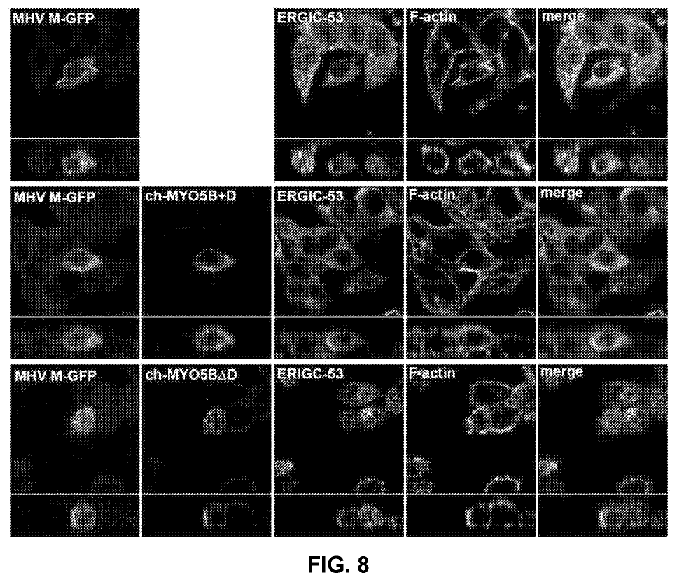

The vast majority of previous studies have used non-polarized cells for analysis of virus infection. However, MDCK cells were used because coronaviruses normally infect polarized epithelial cells at points of primary entry in multiple organ systems. To determine if the localization findings were particular to MDCK cells, colocalization in A549 lung epithelial cells was also evaluated. FIG. 8 demonstrates that, similar to the MDCK cells, coronavirus M proteins colocalized with Cherry-MYO5B+D, but not with Cherry-MYO5BΔD. The M proteins mostly localized with Rab10 and Rab11a, although some localization in the Golgi apparatus was observed. Previous investigations, based on non-polarized cells, have also suggested that coronavirus M proteins were assembled in the ERGIC. While no antibodies are available to establish the distribution of ERGIC in MDCK cells, it was possible to assess localization in the A549cells. Interestingly, little overlap with ERGIC-53 staining was observed (FIG. 8).

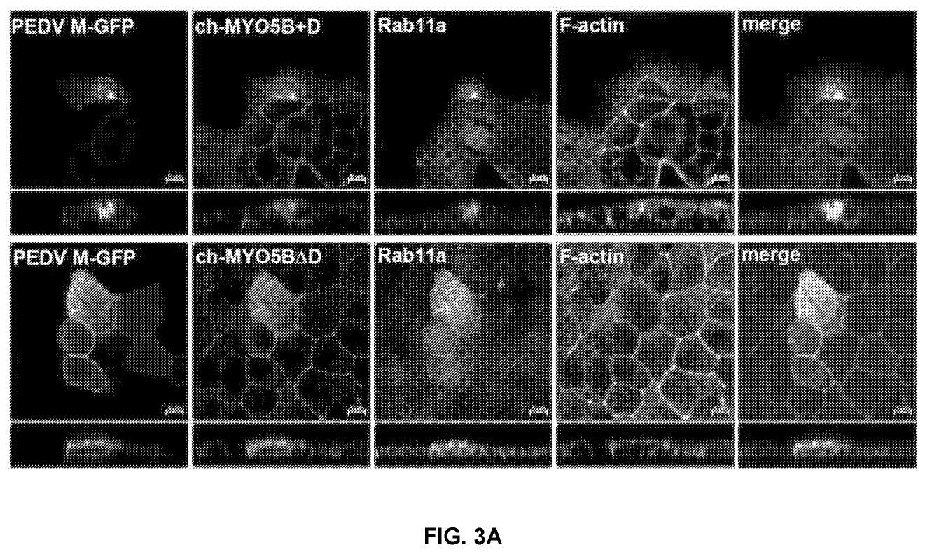

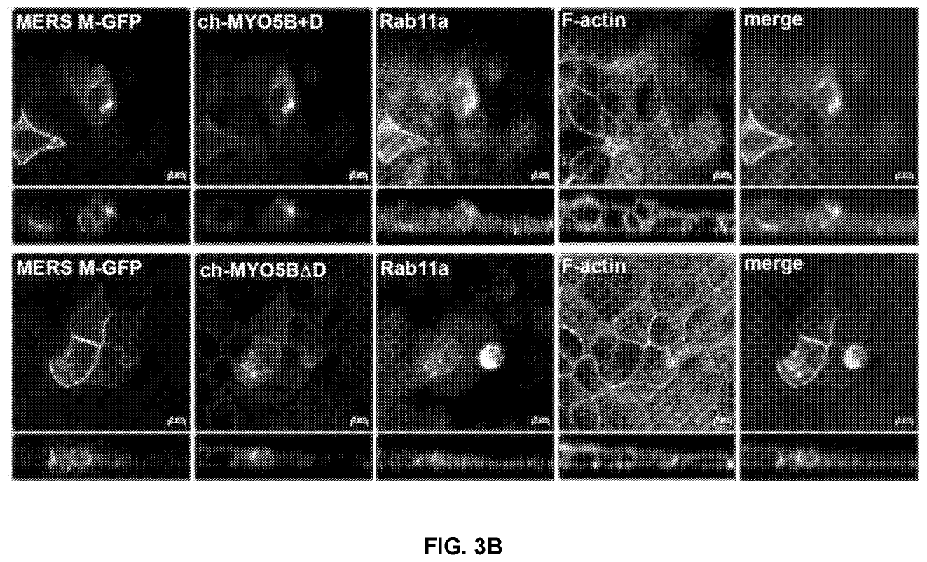

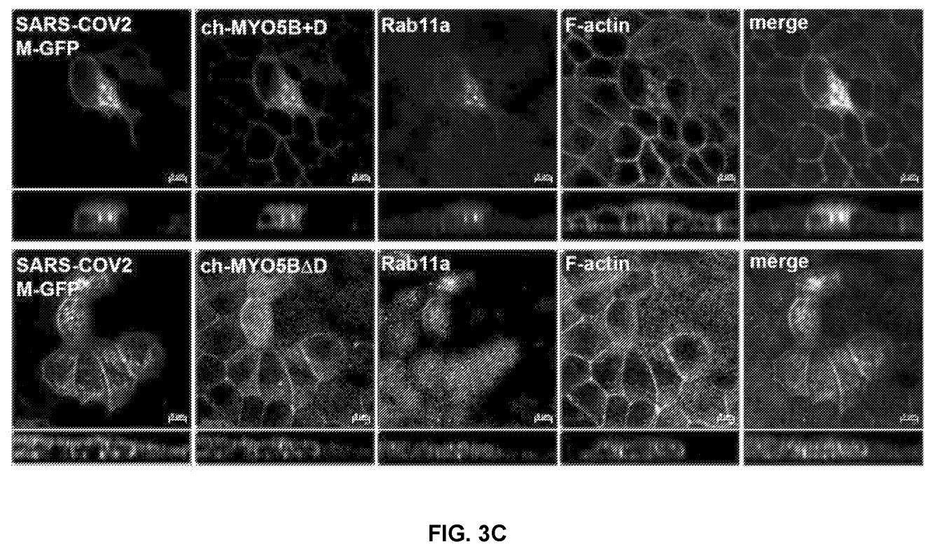

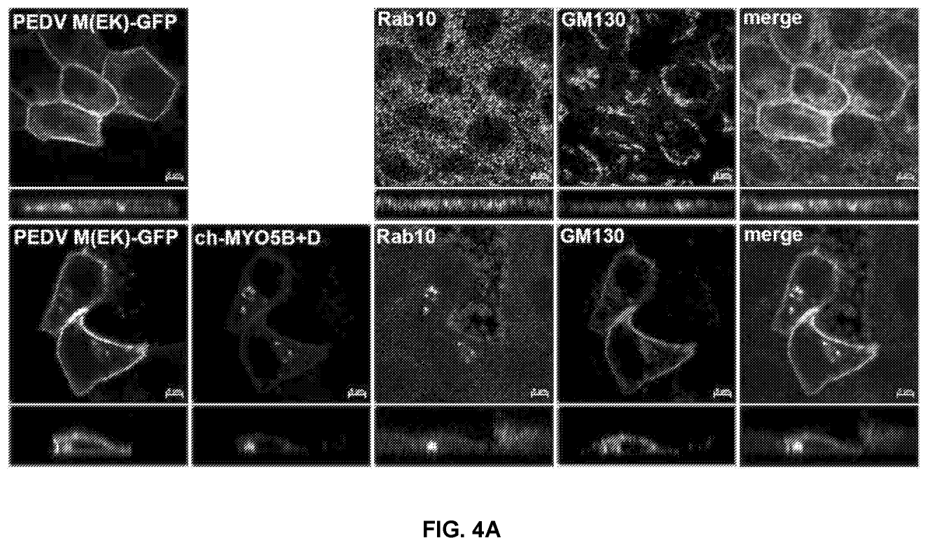

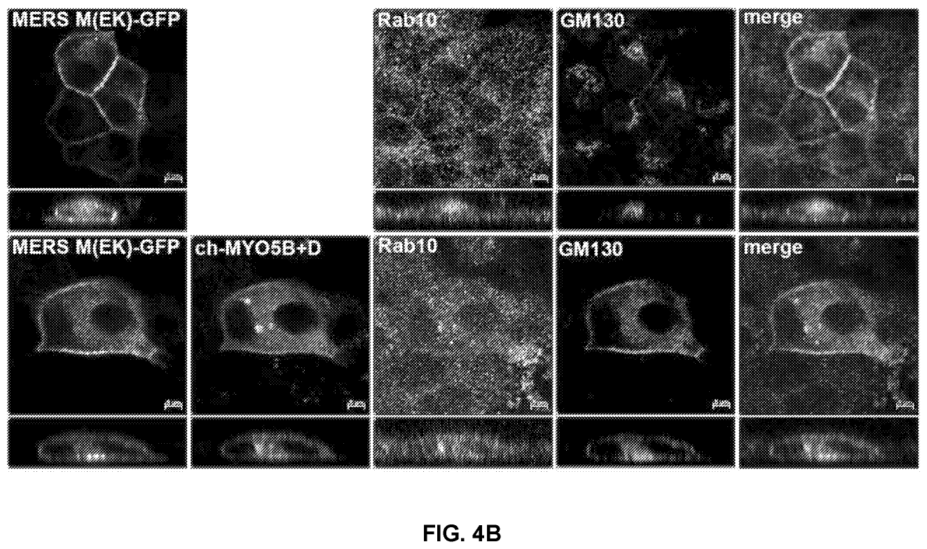

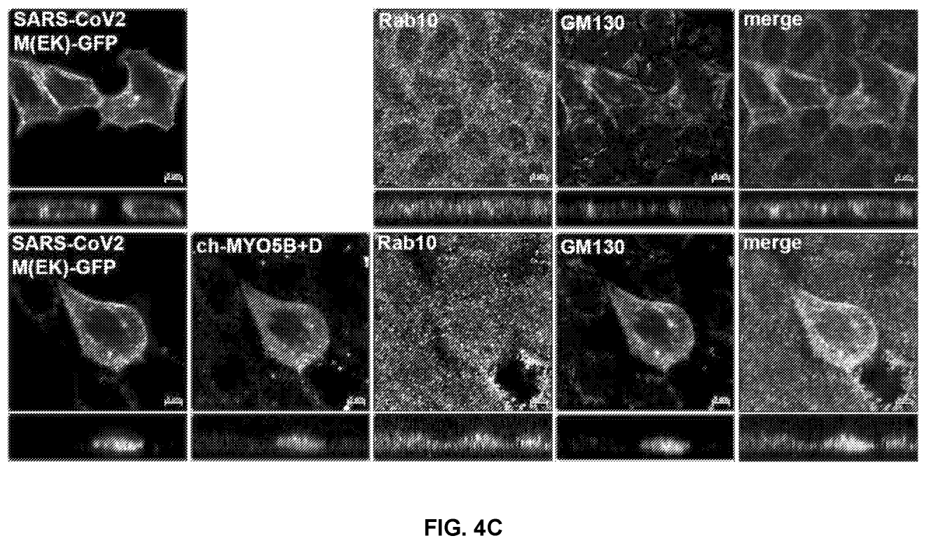

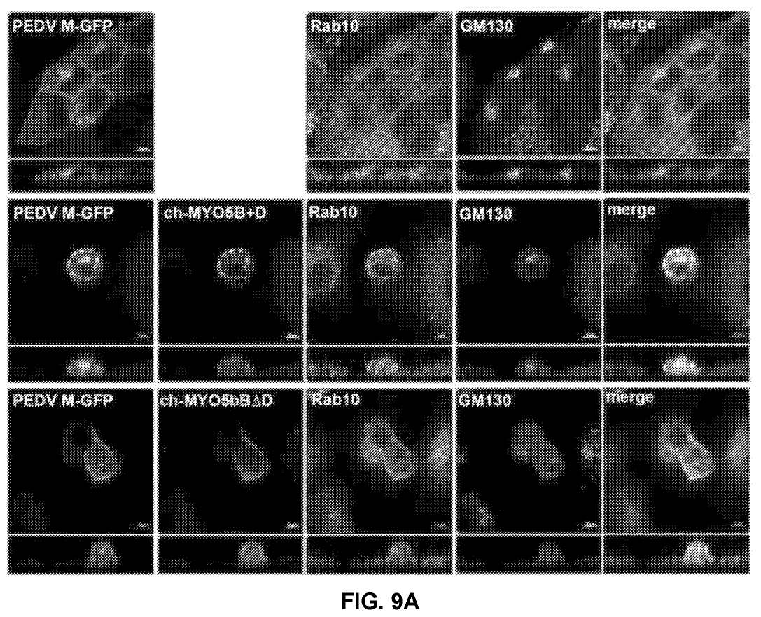

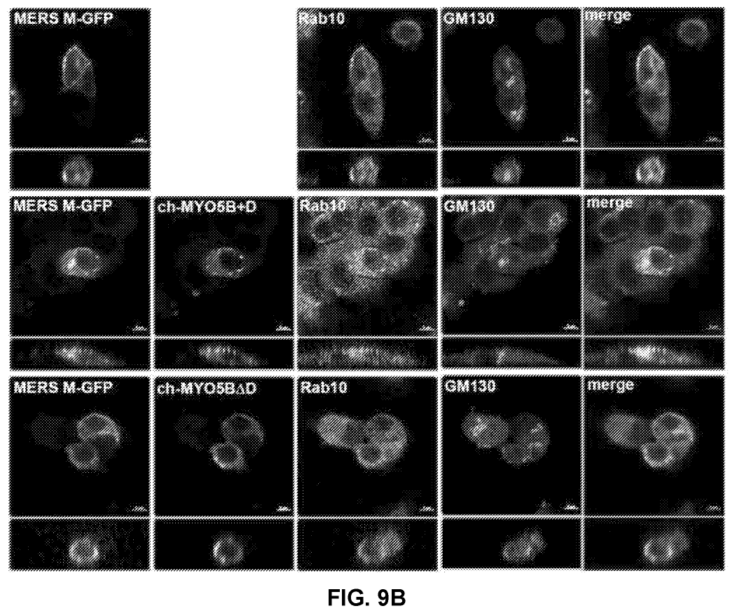

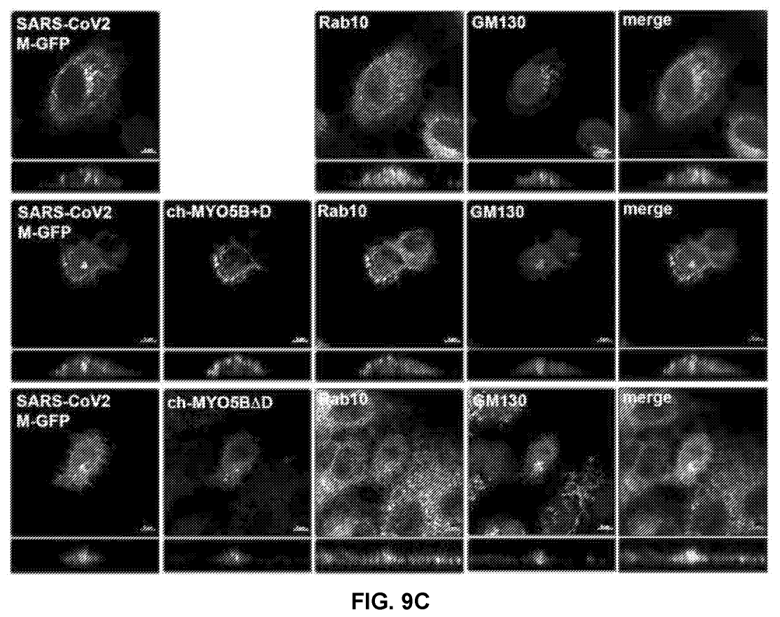

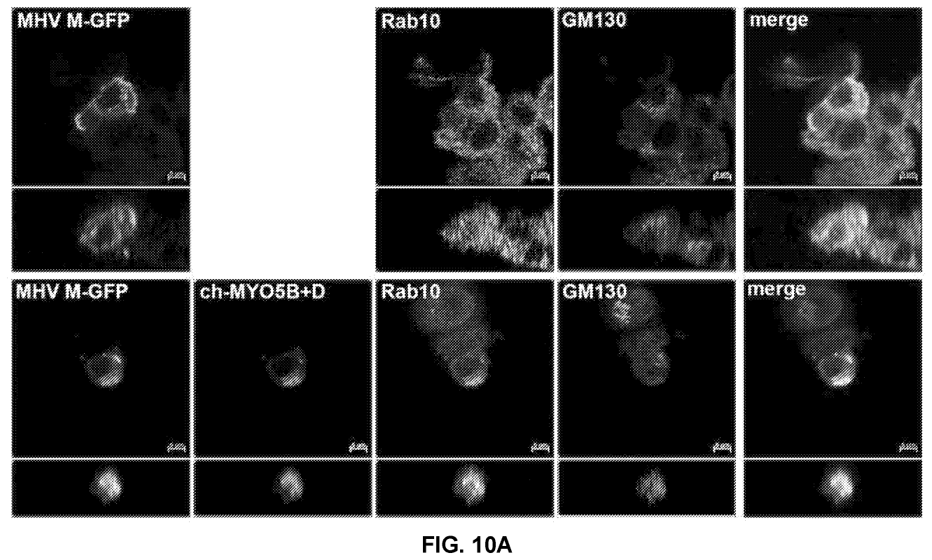

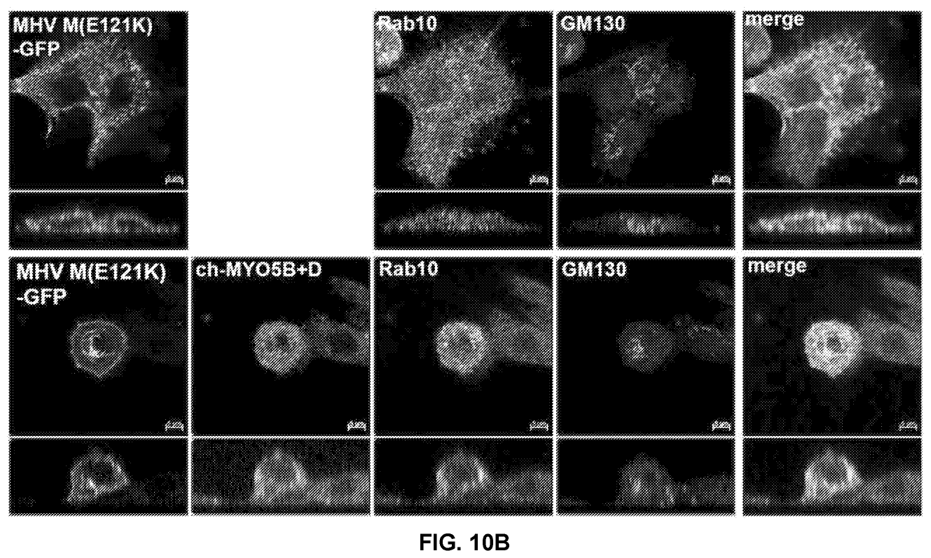

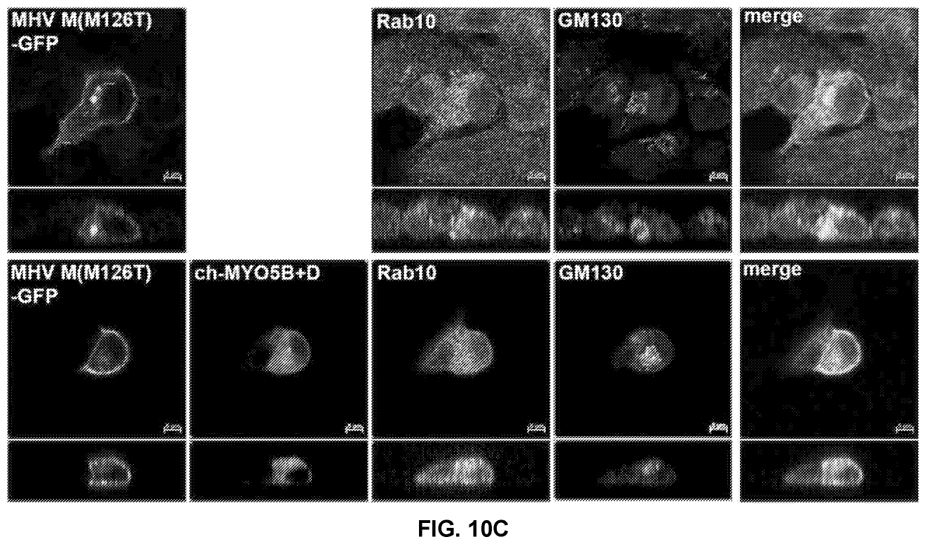

In yeast 2-hybird screens, interactions of other coronavirus M tails with MYO5B(ABCDE) were not definitively identified. Therefore, the localization of M-GFP chimeric proteins for MERS, SARS-CoV-2 and PEDV co-expressed with Cherry-MYO5B+D was assessed (FIG. 3). In all three cases, there was colocalization between the M proteins and MYO5B+D. Nevertheless, Manders' coefficients demonstrated that little co-localization was observed when M proteins were expressed with Cherry-MYO5BΔD (FIGS. 3, 6B). Similar results were observed for co-expression of coronavirus M protein-GFP chimeras with MYO5B+D in A549 lung cells (FIG. 9). Additionally, mutations of the coronavirus M proteins in positions homologous with the E121K mutation in MHV M also abolished co-localization with MYO5B+D (FIGS. 4, 6C). Again, similar results were obtained with co-transfection studies in A549 cells (FIG. 10).

All of the results here indicate that the relatively rare MYO5B+D splice variant is critical for interaction with coronavirus M proteins. Previous investigations have indicated that MYO5B is a multifunctional regulator of vesicle trafficking through its interactions with Rab11a, Rab11b, Rab25, Rab8a and Rab6a (Lapierre, L A. & Goldenring, J R. Methods Enzymol 2005 403:715-723; Roland, J T., et al. Mol Biol Cell 2007 18:2828-2837; Lindsay, A. J. et al. Mol Biol Cell 2013 24:3420-3434), but only sequences with the 26 amino acid alternatively spliced Exon D can interact with Rab10 (Roland, J T., et al. J Biol Chem 2009 284:1213-1223). While the splice variant without Exon D is ubiquitous, the expression of MYO5B+D is enriched in lung, brain, heart and intestine, all target tissues for coronavirus infection (Roland, J T., et al. J Biol Chem 2009 284:1213-1223). Other viruses, including respiratory syncytial virus (Utley, T J. et al. Proc Natl Acad Sci U S A 2008 105:10209-10214; Brock, S C., et al. Proc Natl Acad Sci U S A 2003 100:15143-15148; Shaikh, F Y. et al. PLoS ONE 2012 7:e40826), influenza (Vale-Costa, S. et al. J Cell Sci 2016 129:1697-1710), Epstein Barr Virus (Nanbo, A. Microorganisms 2020 8), and HIV (Qi, M. et al. PLoS Pathog 2013 9:e1003278; Varthakavi, V. et al. Traffic 2006 7:298-307) utilize component regulators of the plasma membrane/apical membrane recycling systems to accomplish virus assembly and trafficking in host cells. The present data suggest that, in polarized epithelial cells, the apical recycling system may be a site for assembly of coronavirus virus particles. M proteins accumulated with MYO5B+D in vesicles containing Rab11a and Rab10. Rab11a is strongly associated with the apical recycling system in polarized cells (Casanova, J E. et al. Mol Biol Cell 1999 10:47-61; Wang, X., et al. J. Biol. Chem. 2000 275:29138-29146), although Rab11a can also be found in association with the Golgi apparatus (Urbe, S., et al. FEBS Lett. 1993 334:175-182). Rab10 has been associated with both endosomal and exocytotic pathways in different cell systems (Etoh, K. & Fukuda, M. J Cell Sci 2019 132; Chua, CEL. & Tang, B L. J Cell Physiol 2018 233:6483-6494). While the previous studies in non-polarized cells had suggested that coronavirus virions were assembled in ERGIC, in polarized epithelial cells, the recycling system may play a more prominent role.

In summary, these studies have identified MYO5B+D as a critical host protein interactor with coronavirus M proteins. The ability of an E121K mutation in MHV M to disrupt binding to MYO5B+D was also shown. Since previous studies have identified this same mutation as blocking the assembly of both virus like particles as well as whole virion assembly (Arndt, A L., et al. J Virol 2010 84:11418-11428), it seems reasonable to suggest that MYO5B+D may play a critical role in M protein trafficking in host cells and its interaction with M protein is likely necessary for efficient viral assembly. Since this association between M proteins and MYO5B+D appears conserved among beta-coronaviruses, these finding make interruption of the interaction between coronavirus M proteins and MYO5B+D a prominent target for impacting the deleterious effects of coronavirus infection generally in present as well as future species.

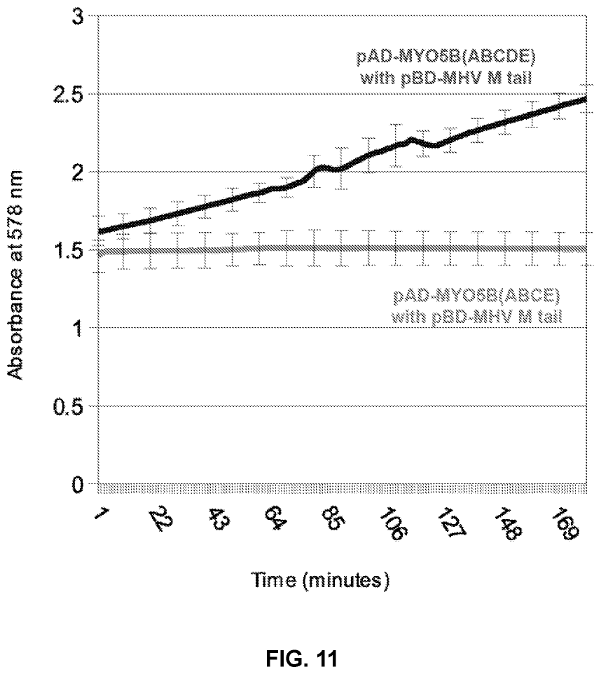

The yeast 2-hybrid assay provides a reliable method for assessing the interaction of MHV M tail with MYO5B+D. A screen was performed of small molecules that might be able to block the interaction using a 1800 compound library of approved drugs. The assay utilized yeast harboring both pBD-MHV M tail and pAD-MYO5B(ABCDE) which were grown in the presence of 10 μM concentrations of small molecules for 24 hours. The loss of b-galactosidase activity indicative of a loss of interaction between bait and prey constructs was assessed. Yeast cultures were grown in 96 well microtiter plates in media lacking leucine and tryptophan. Potential toxicity of the drugs was assessed by determining culture densities in each well at OD600 and observation of an intact yeast pellet. Drugs that induced yeast death were excluded from the analysis. Yeast were lysed in the wells in the presence of β-galactosidase substrate (chlorophenol red-β-D-galactopyranoside, CPRG) and the activity was assessed in a microtiter plate reader (578 nm).68, 69 Yeast harboring the pair of pBD-MHV M tail and pADMYO5B(ABCE) were used as a negative control to establish a baseline (FIG. 11). Note that the linear β-galactosidase activity out to 180 min allows strong separation of signal from noise. Hit compounds were selected that showed >75% loss of b-galactosidase activity without significant effects on yeast growth. The FDA-approved drug library (1800 compounds) was dispensed into 96 well plates by the Vanderbilt High Throughput Screening (VHTS) Shared Resource.

Out of the initial screen, 5 compounds were identified as hits: Amiodorone, Diclofenac, Nimesulide, Nizatidine and Nomifensine. The effects were confirmed in 3 repeat yeast 2 hybrid assays.

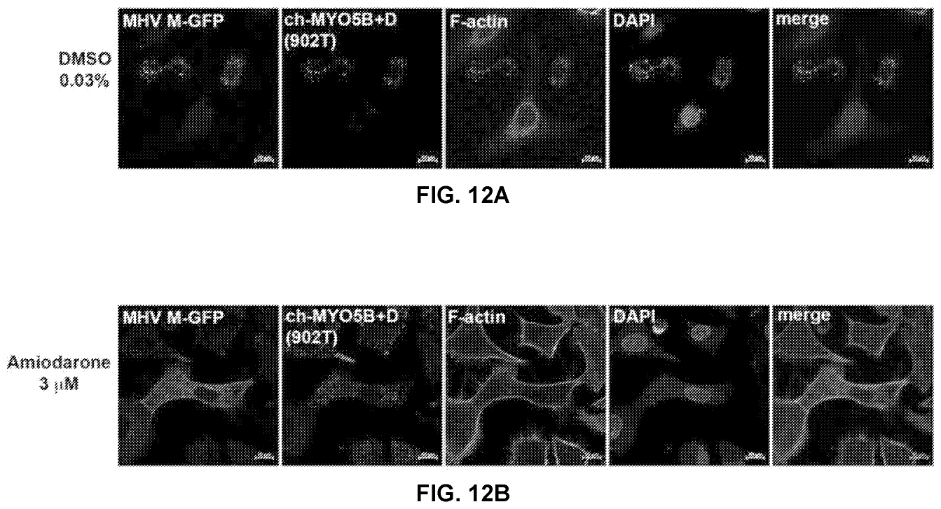

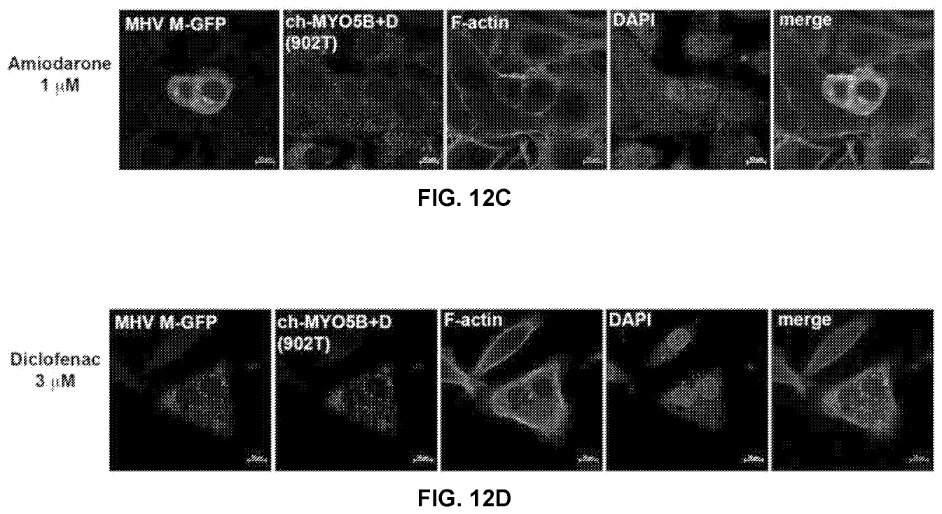

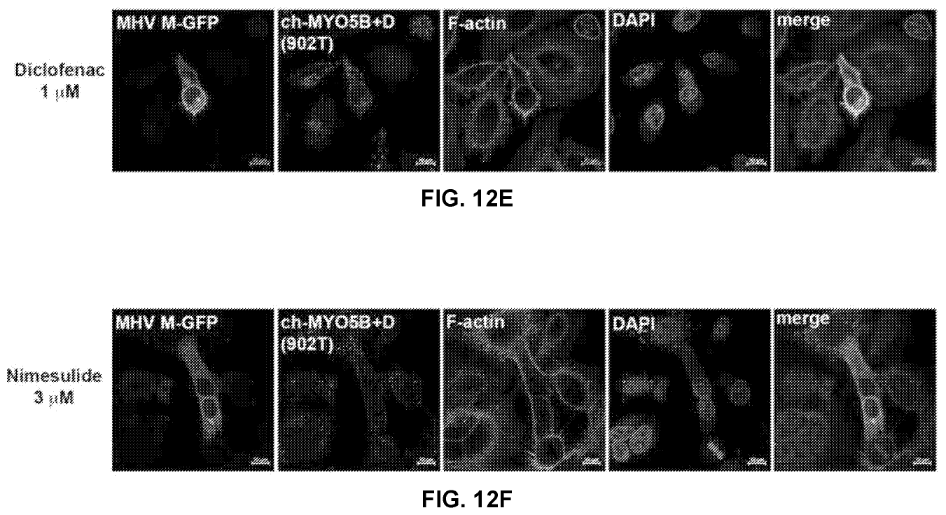

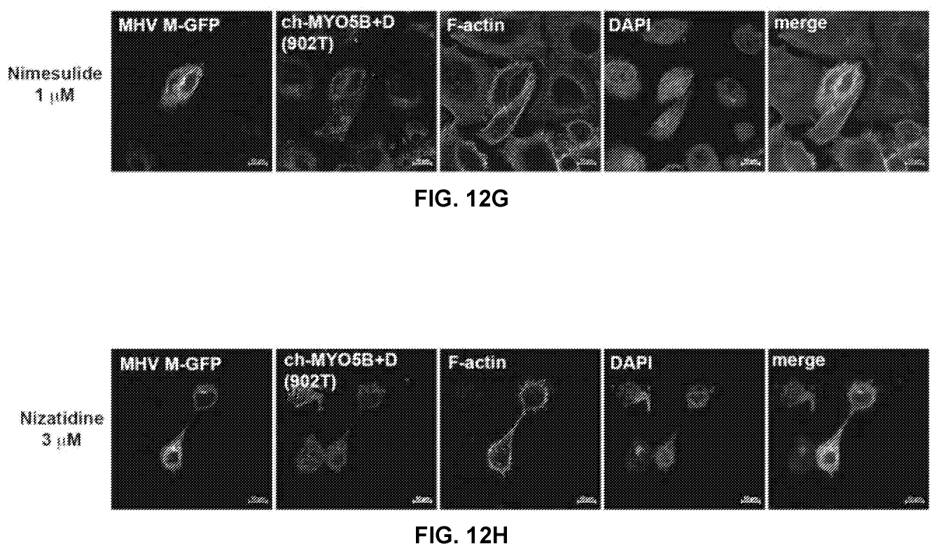

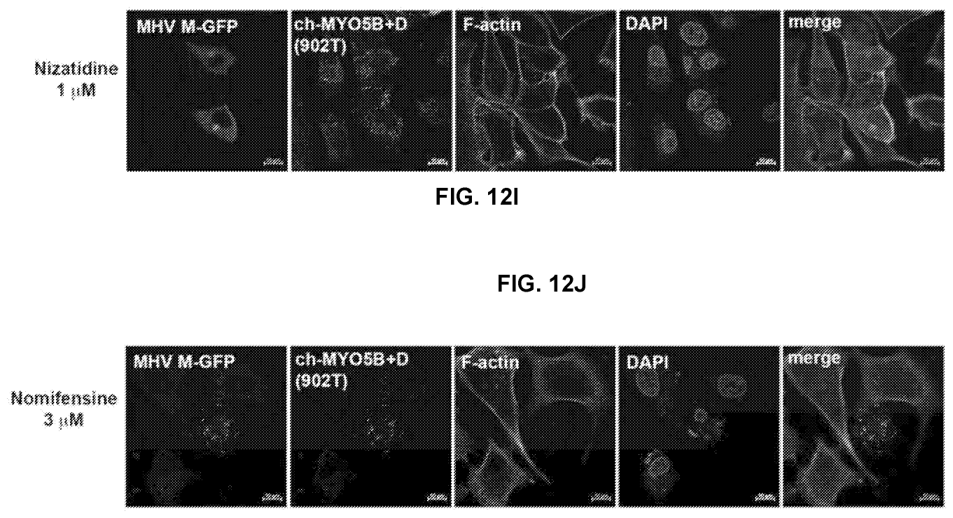

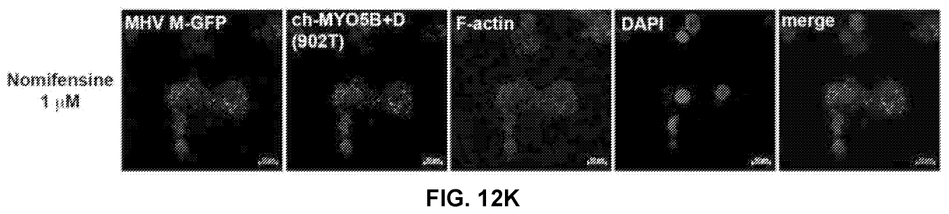

As a secondary screen to test the efficacy of the compounds, A549 lung cells co-expressing MHV M-GFP and Cherry-MYO5B+D tail (amino acids 902-1848) were cultured in the presence of 3 and 1 μM drug for 24 hours and fixed cells were stained for F-actin (fluorescent-phalloidin) and DAPI (FIG. 12A-12K). The representative images in FIG. 2 show that Amiodorone and Nizatidine, showed inhibition of the interaction between MHV M and MYOS5B+D at both 3 and 1 μM. Diclofenac and Nimesulide showed more equivocal effects at 1 μM. Nomifensine did not alter the interaction at either dose.

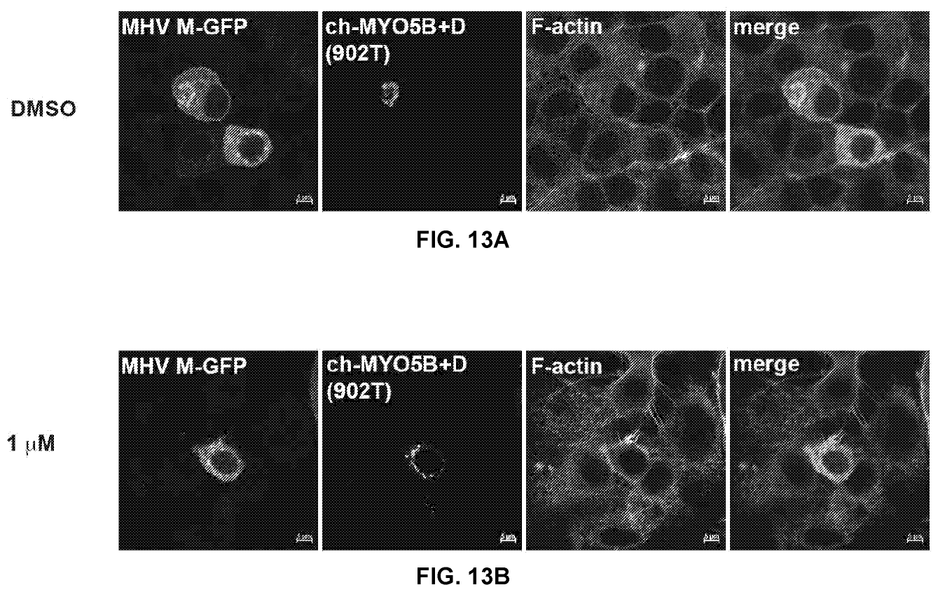

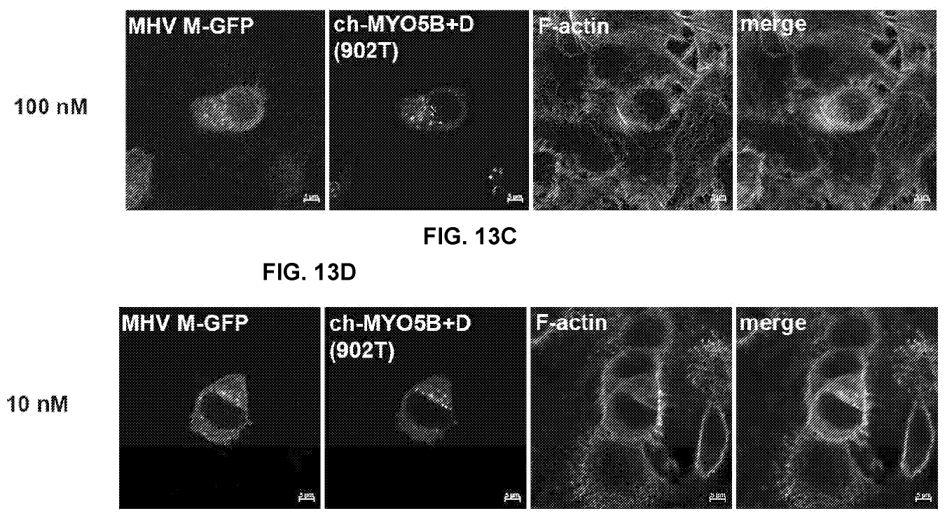

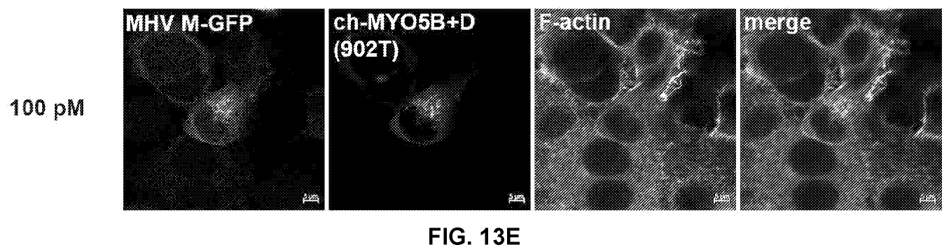

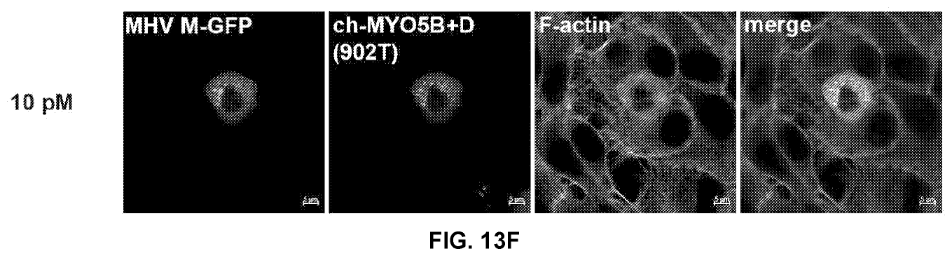

Because of the relative strength of the effects of Nizatidine, the interaction of MHV M-GFP and Cherry-MYO5B+D tail was next evaluated in cells exposed to a range of nizatidine doses from 10 pM to 1 μM for 24 hours. FIGS. 13A to 13F demonstrates that nizatidine at doses from 1 μM down to 100 pM disrupted the co-localization of MHV M with MYO5B+D.

Based on these initial findings it appears that nizatidine represents a potent inhibitor of the interaction of MHV M with MYO5B+D. The ED₅₀ for nizatidine actine as an inhibitor of the H2-histamine receptor is 0.7 nM. This suggests that nizatidine may be able to disrupt the assembly of MHV and other coronaviruses.

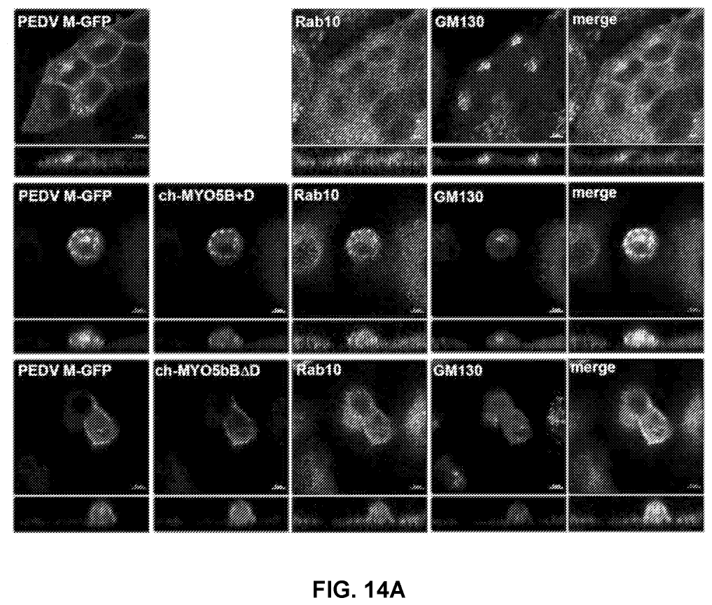

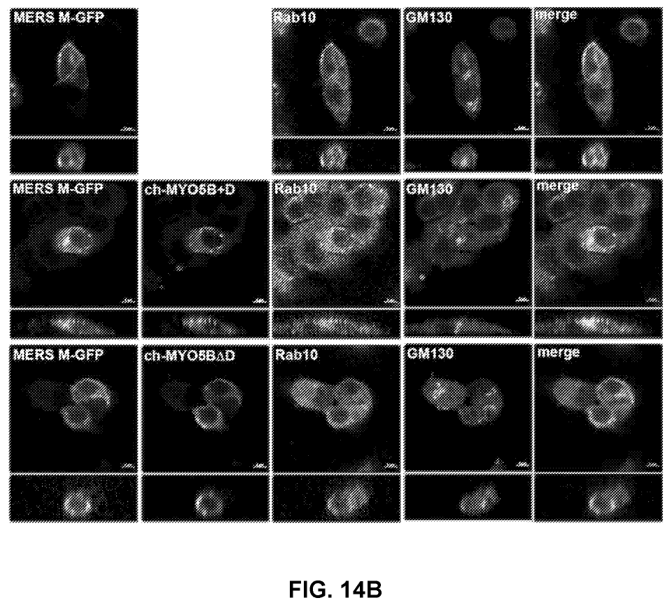

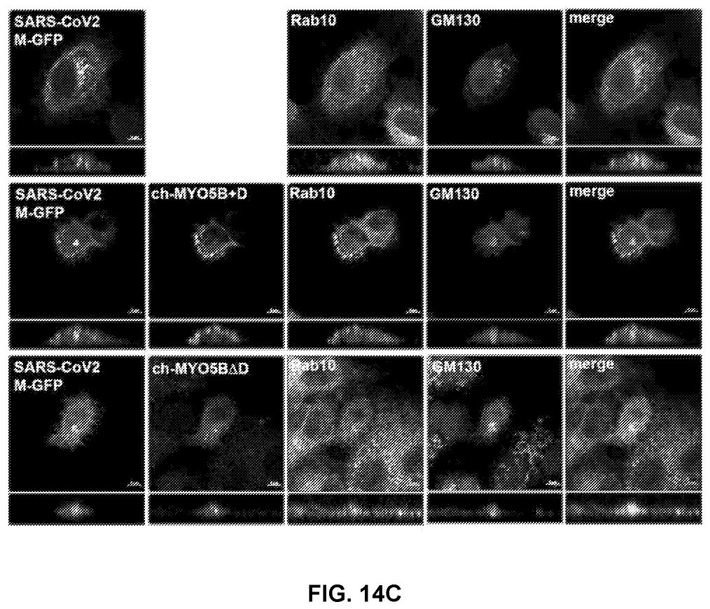

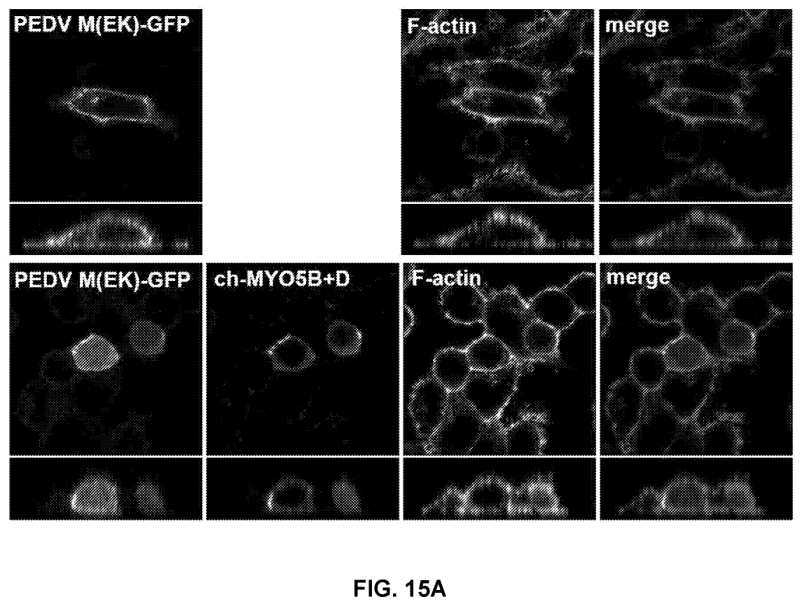

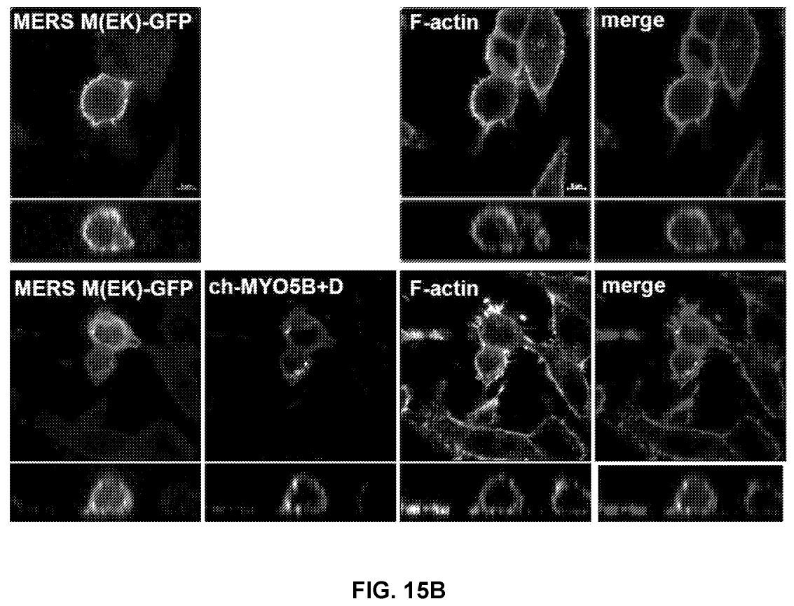

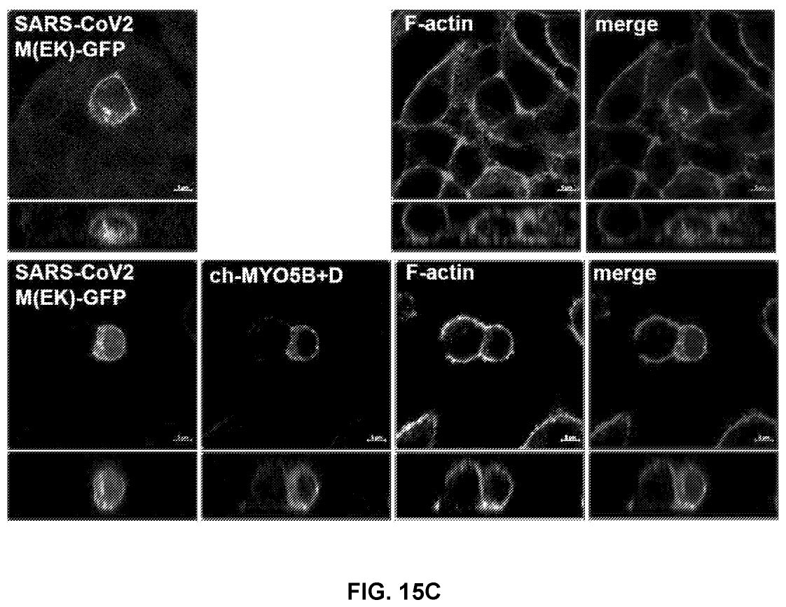

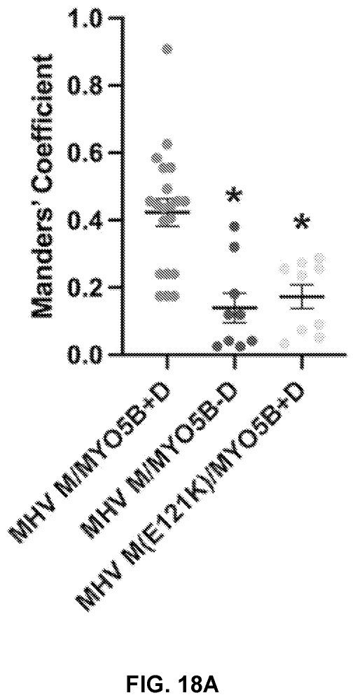

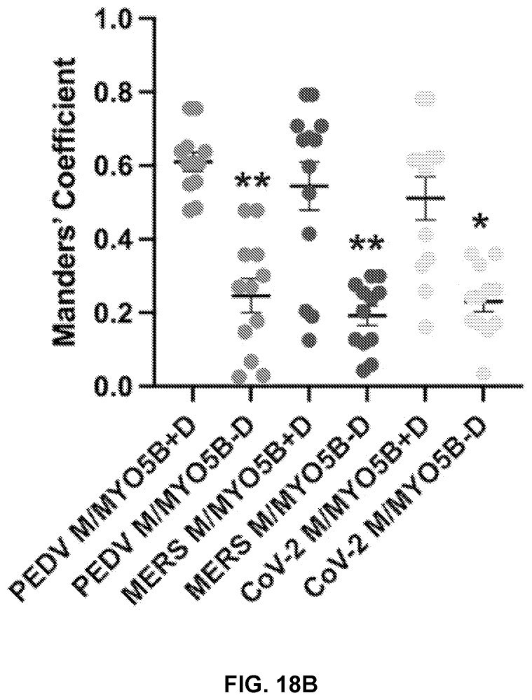

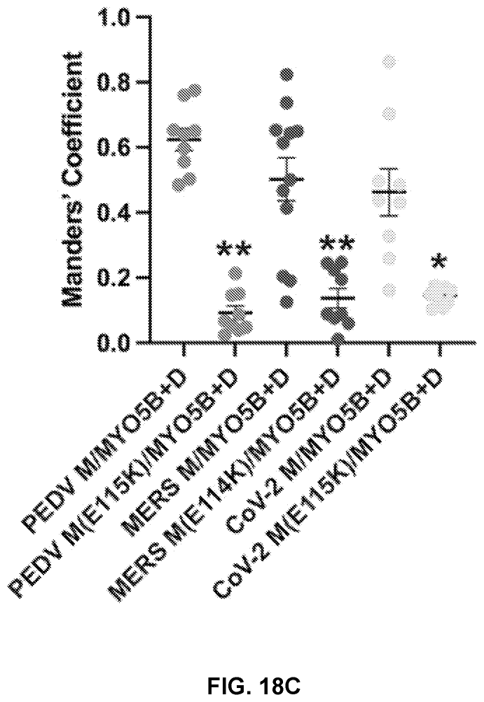

Based on the findings in the split-ubiquitin yeast 2-hybrid studies, we next assessed the localization of M-GFP chimeric proteins for PEDV, MERS and SARS-CoV-2 coexpressed with Cherry-MYO5B+D in MDCK cells (FIGS. 3A to 3C). In all three cases, there was colocalization between the M proteins and co-expressed Cherry-MYO5B+D in an internal vesicular compartment. In addition, Manders' coefficients demonstrated that co-localization was markedly disrupted when M proteins were expressed with Cherry-MYO5BDD (FIGS. 3A to 3C, and 6B). Similar results were observed for coexpression of coronavirus M protein-GFP chimeras with MYO5B+D in A549 lung cells (FIGS. 14A-14C and 18B). All coronavirus M proteins contain a glutamate residue in a homologous position to the E121 residue in MHV M.16 Mutation of this glutamate residue to lysine in all of the coronavirus M proteins abolished co-localization with MYO5B+D (FIGS. 4A to 4C, and 6C) in MDCK cells. Similar results were also found with the coronavirus M protein E to K mutants expressed in A549 cells (FIGS. 15A-15C and 18C).

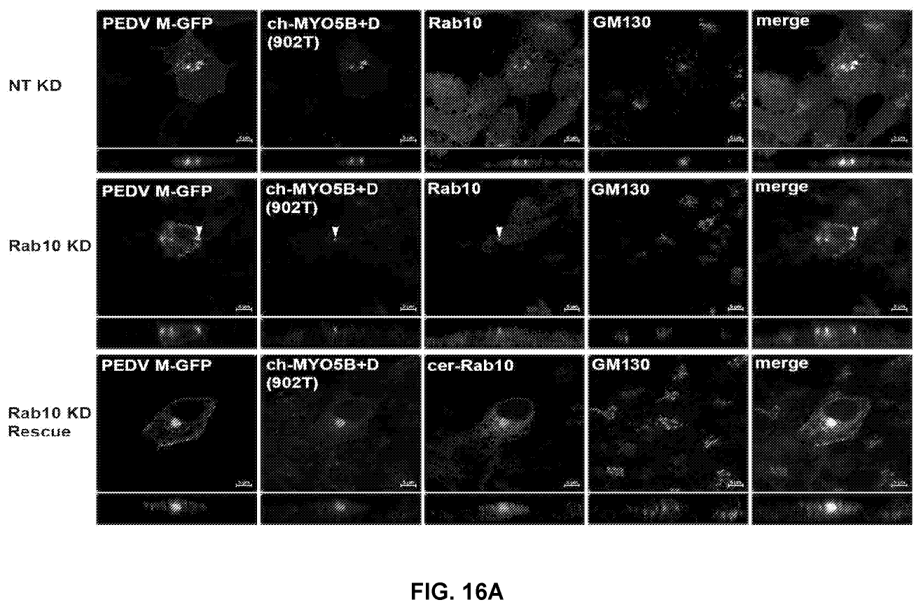

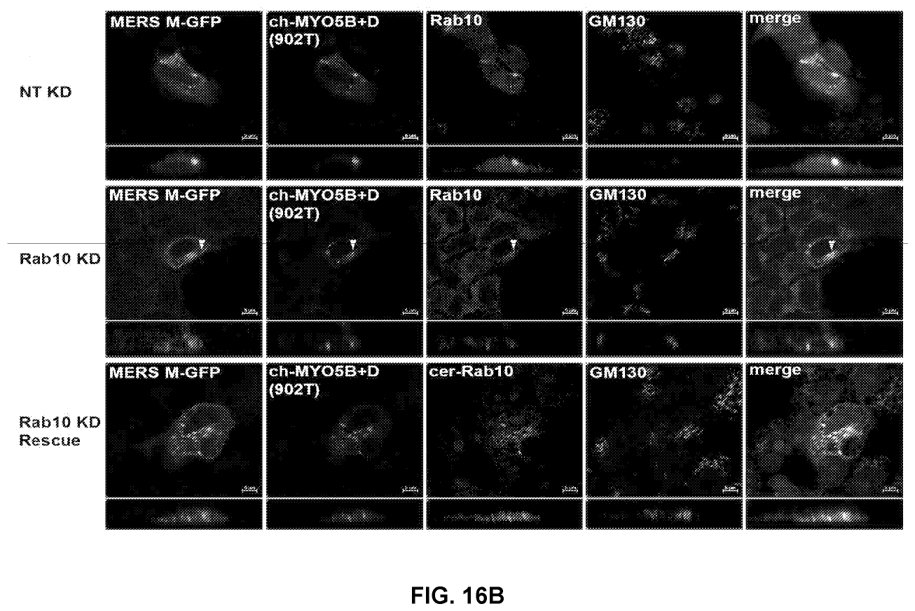

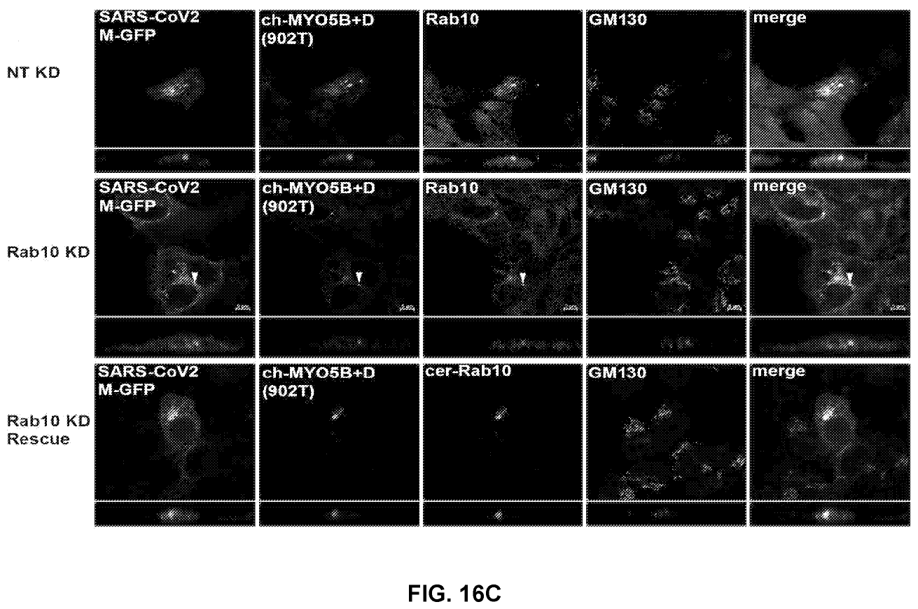

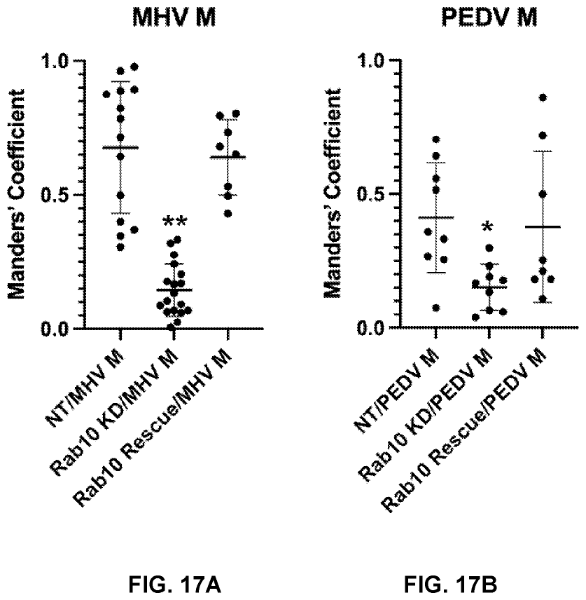

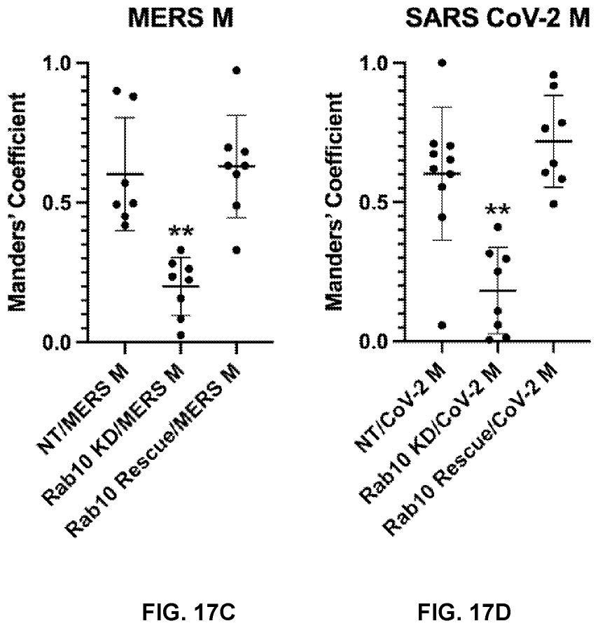

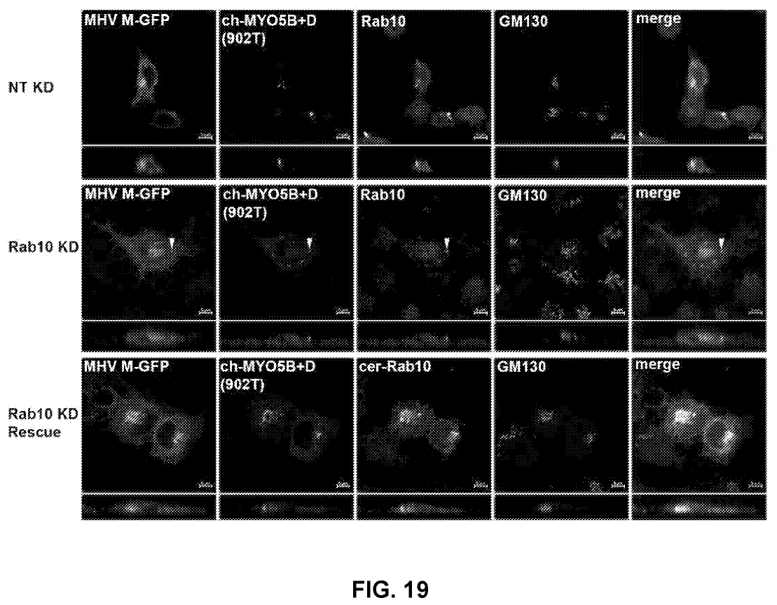

The D exon of MYO5B is responsible for interactions with Rab10.14 A motorless tail construct of MYO5B can cause concentration of MYO5B with binding Rab proteins in a collapsed membranous cisternae. Therefore, next examined was whether knockdown of Rab10 could alter the trafficking of coronavirus M proteins. FIG. 19 demonstrates expression of a mCherry-MYO5B+D tail construct (902T) in A549 cells caused concentration of MHV M protein with endogenous Rab10. Dual transfected cells also showed a more diffuse pattern of Golgi apparatus staining. Nevertheless, in A549 cells with knockdown of Rab10, we observed a loss of colocalization of MHV M with MYO5B+D tail (FIGS. 17A to 17D and 19). Reintroduction of Cerulean-Rab10 resistant to shRNA, re-established the colocalization of MYO5B+D tail with MHV-M. FIGS. 16A to 16C demonstrate that similar sequestration of M proteins was observed for M proteins from MERS, PEDV and SARS-CoV2 with expression of a mCherry-MYO5B+D tail. As for MHV M, knockdown of Rab10 expression led to a loss of colocalization of MYO5B+D and M proteins (FIGS. 16A-16C and 17A to 17D). Re-expression of Cerulein-Rab10 re-established colocalization of M proteins with MYO5B+D tail.

### Materials and Methods

### DNA Sequence Construction

DNA sequences for full length and cytoplasmic tails of M proteins PEDV, MERS and SARS-CoV-2 were synthesized with appropriate flanking restriction sites by GeneArt (Thermo-Fisher). M protein cytoplasmic tails were ligated into pBD-Gal(Cam) following restriction digest and gel isolation. Full-length M protein sequences were ligated in pEGFP-N1 following restriction digest and gel isolation. Site-directed mutagenesis of M protein sequences was performed in all cases using a single nucleotide mutagenesis protocol (see Table 1 for sequences) utilizing the Quickchange Lightening Multi Site-directed Mutagenesis Kit (Agilent, Santa Clara, CA). All mutagenized sequences were confirmed using Sanger sequencing (GenHunter, Nashville, TN). The sequences of human MYO5B+D 902 tail and MYO5BDD 902 tail were inserted into pmCherry-C1 vector using flanking BamHI and SaII restriction sites.

### Yeast Two-Hybrid (Y2H) Screen

The Y2H screen was performed following a protocol previously described (Vidalain, P. O. et al. Methods Mol Biol 1282:213-229 (2015)). The DNA sequence encoding the C-terminal cytoplasmic tail of membrane protein (Mcyto) of mouse hepatitis coronavirus strain A59 (starting with S105) was cloned by in vitro recombination (Gateway technology; Invitrogen, Carlsbad, CA, USA) from pDONR207 into the Y2H vector pPC97-GW (kindly provided by Dr. Marc Vidal) for expression in fusion downstream of the GAL4 DNA-binding domain (GAL4-BD). AH109 yeast cells (Clontech; Takara, Mountain View, CA, USA) were transformed with the GAL4-BD-Mcyto plasmid using a standard lithium acetate protocol. Transactivation of the HIS3 reporter gene by the GAL4-BD-Mcyto protein alone was tested on culture medium lacking histidine, and concentration of 3-amino-1, 2, 4-triazole (3-AT) was adjusted to prevent spontaneous yeast growth in the absence of interacting prey protein during the screen. In parallel, a mouse brain cDNA library cloned into yeast two-hybrid vector pPC86 (Life Technologies) to express prey proteins in fusion downstream of the GAL4 transactivation domain (GAL4-AD) was transformed into Y187 yeast cells (Clontech; Takara, Mountain View, CA, USA). Finally, AH109 cells expressing GAL4-BD-Mcyto were mated with Y187 cells expressing the prey cDNA library and were plated on -L-T-H+3AT selective medium. After six days of culture, colonies were picked and replica plated over three weeks on selective medium to eliminate potential contamination with false positives. Prey proteins from selected yeast colonies were identified by PCR amplification using primers that hybridized within the pPC86 regions flanking the cDNA inserts. PCR products were sequenced and cellular interactors identified by BLAST analyses.

### Binary Yeast Two-Hybrid Assays

DNA sequences to be screened were cloned into either the pAD activation vector or the pBD bait vector (Clontech). For yeast 2-hybrid assays, a culture of Y190 yeast was grown overnight and diluted to OD₆₀₀=0.2 in 2 mL YPDA media per reaction, then allowed to grow to log phase. Yeast were pelleted at 3000×g for 3 minutes. Cells were resuspended in 20 μL per sample 0.1 M lithium acetate in TE buffer (10 mM Tris-HCl, 1 mM EDTA, pH 7.5). This solution was added to 120 μL per sample 0.1 M lithium acetate/40% PEG 3350 in TE buffer and mixed by inversion. Each pAD and pBD vector was aliquoted to 1 μg, then 135 μL of transformation solution was added to the vectors for each reaction and incubated for 30 minutes at 30° C. with shaking at 75 rpm. DMSO was added to each reaction to 10% and reactions were incubated for 15 minutes at 42° C. Yeast were pelleted at 2000×g for 3 minutes, supernatant was removed and yeast were resuspended in 30-50 μL H₂O and plated on Trp/Leu drop-out plates. After 3 days of growing at 30° C., a colony lift assay was performed. Filter paper was pressed onto colonies to lift them from the plate, then the filter paper was frozen for 20 seconds in liquid nitrogen. Filter papers were thawed for several minutes, then frozen in liquid nitrogen again for 20 seconds. After several minutes of thawing, 2 mL 0.33 mg/mL X-gal (from 20 mg/mL solution in DMF) in Z-buffer (60 mM Na₂HPO₄-7H₂O, 40 mM NaH₂PO₄-H₂O, 10 mM KCl, 1 mM MgSO₄-7H₂O, pH 7) was dispensed on a clean filter paper, then each filter paper with frozen colonies was placed onto a wet filter paper to absorb the Xgal solution. Colony color change was observed every 30 minutes for approximately 3 hours. All binary yeast 2-hybrid assays were repeated at least 3 times to identify consistent results.

### Random Mutagenesis

For the random mutagenesis screen, a PCR reaction was performed on selected DNA inserts in pBD vectors using Qiagen Taq polymerase (201203). The reaction was composed of 0.2 μg pBD vector with MHV M cytoplasmic tail insert, 1×Qiagen PCR buffer (201203), 0.2 mM dNTPs, 1 μM each sense and anti-sense primer, and 1% Taq polymerase with a total volume of 100 μL. The reaction was heated for 5 minutes at 94° C., then run for 10 cycles of 30 seconds at 94° C., 30 seconds at 58° C., and 1 minute per 1000 base pairs at 72° C., then heated for 5 minutes at 72° C. MnCl₂ was added to the reaction to 5 μM, then the reaction was run for 30 cycles of 30 seconds at 94° C., 30 seconds at 58° C., and 2 minutes at 72° C., then heated for 5 minutes at 72° C. The PCR product was purified using the QiaQuick PCR Purification Kit (28104), the mutated insert was cut out of the pBD vector, gel-isolated, extracted using the QiaQuick Gel Extraction Kit (28704), and ligated into empty pBD vector.

The pBD vector library with mutagenized MHV M cytoplasmic tail insert was screened against target pAD-MYO5B(ABCDE) vector by transforming the vectors into yeast as described above and performing a blue-white b-galactosidase assay. Colonies that stayed white in the blue-white assay were grown up in YPDA media for 3 days, then vectors were harvested using the QiaPrep Spin Miniprep Kit (27104) with the addition of 5 minutes of vortexing yeast with approximately 100 μL of glass beads after adding resuspension buffer. The resulting vectors were transformed into DH5α cells and grown on chloramphenicol plates to select for the pBD vectors with mutagenized inserts. Vectors were harvested from the bacterial colonies and sequenced using Sanger sequencing. All putative mutant sequences were confirmed with binary yeast 2-hybrid assays using rescued vector sequences.

### Split-Ubiquitin Yeast 2-Hybrid Assays

DNA sequences for full length M proteins were cloned into pCCW bait vector and MYO5B exons ABCDE sequences were cloned in the pDSL prey vector or the (Dualsystems Biotech). For split-ubiquitin assays, a culture of Nym32 yeast was grown overnight in YPDA media and diluted to OD₆₀₀=0.2 in 2 mL YPDA media per reaction, then allowed to grow to log phase. Yeast were pelleted at 3000×g for 3 minutes. Yeast were resuspended in 100 μL H₂O per sample, then added to tubes containing 290 μL of transformation solution (5% salmon sperm DNA previously heated to 100° C./40% PEG 3350/0.12 lithium acetate) and 1 μg of the appropriate pDSL and pCCW vector for each transformation. The transformation reactions were mixed by vortexing for 20 seconds, then were incubated for 45 minutes in a 42° C. water bath. Yeast were pelleted at 2000×g for 3 minutes, supernatant was removed and yeast were resuspended in 30-50 μL H₂O and plated on -Trp/-Leu plates.

After 3 days of growing at 30° C., a spot assay was performed using the transformed yeast. Yeast from each transformation were resuspended in 500 μL H₂O and diluted to the same OD₆₀₀ between 2 and 5 depending on each experiment. Yeast were then diluted 1:10, 1:50 and 1:100, and 5 μL of each dilution (including the original 1× dilution) were spotted onto -Trp/-Leu/-His and -Trp/-Leu/-His+10 mM 3-amino-1,2,4-triazole (Sigma-Aldrich) plates, then air-dryed. Yeast were grown for 3 days at 30° C., then plates were photographed.

Yeast growth was rated on a scale of 0-8 in which 0 was no growth, 1 was a few colonies in the 1× spot, 2 was a solid dot in the 1× spot, 3 was a solid dot in 1× and a few colonies in the 1:10 spot, 4 was solid dots on 1× and 1:10, and so forth for all 4 dilutions.

### Immunoprecipitation and Western Blotting

Cells expressing a MHV M-GFP and a cherry-MYO5B+D construct grown on 6-well plates were washed in ice cold PBS, scraped, pelleted, and lysed in 1% CHAPS/0.5 mM EDTA/TBS with mammalian protease inhibitor cocktail (Sigma) for minutes with endover-end tumbling at 4° C. Lysates were spun for 10 minutes at 13,000×g at 4° C., then brought to 500 μl in dilution buffer (10 mM Tris/Cl pH 7.5/150 mM NaCl/0.5 mM EDTA). 10 μl of RFP-Trap Dynabeads (Chromotek) were washed in dilution buffer and added to the lysates, then lysates were incubated for 1 hour with end-over-end-tumbling at 4° C. Beads were separated and washed in wash buffer (10 mM Tris/Cl pH 7.5/150 mM NaCl/0.05% NP-40/0.5 mM EDTA) 3 times for 5 minutes, then resuspended in 20 μl wash buffer with 1× sample buffer. Sample buffer was also added to 20 μl of original lysate from each sample, and beads and original lysates were heated for 2 minutes at 37° C. Original lysates and eluents were run on 4-12% polyacrylamide gels, then transferred onto Odyssey nitrocellulose membrane (LiCor). Membranes were blocked with Odyssey PBS blocking buffer (LiCor), probed with mouse anti-GFP and rabbit anti-RFP antibodies (see Table 2), and then probed with Odyssey anti-mouse 680 nm and anti-rabbit 800 nm secondary antibodies (LiCor). Blots were imaged using a LiCor Odyssey Fc.

### Cell Culture and Transfections

Madin-Darby Canine Kidney (MDCK) cells and the A549 human epithelial lung cell line (Foster, K. A., et al. Exp Cell Res 243:359-366 (1998)) (ATCC) for immunofluorescence and HEK293FT cells for western blotting were plated onto coverslips or 6-well plates and transfected with the indicated vectors using PolyJet (SignaGen Labs). For co-transfection for immunofluorescence, the ratio of vectors for mCherry N-terminally tagged MYO5Bs to the EGFP C-terminally tagged Coronavirus M proteins was 2:1.

For the Rab10-knockdown cell lines, a lentiviral pLK0.1 shRNA vector targeting human Rab10 (Sigma Aldrich TRCN0000382083) and a lentiviral pLKO.5 non-target shRNA control vector (Sigma-Aldrich SHC216-1EA) were used for transduction of A549 MHVR cells. HEK 293FT cells were plated on T75 flasks and grown to ˜50% confluence. To transfect the HEK 293FT cells, 30 μL of Polyjet (SignaGen Labs) transfection reagent per flask was used with a mixture of DNA consisting of 4 μg of the Rab10 targeting or control non-targeting vector, 4 μg of the packaging plasmid psPAX2, and 2 μg of the ENV plasmid pMD2.G. The HEK 293FT cells were refed after 24 hours and incubated for an additional 48 hours. The medium from the cells was then filtered, incubated overnight with the addition of LentiX Concentrator (Takara) 1:3, spun down for 45 minutes at 1500×g, resuspended in media and frozen until use. Concentrated virus was used with polybrene at 5 μg/ml to transduce plated A549 MHVR cells at 30% confluence. After 48 hrs, the A549 MHVR cells were then selected in medium containing 4 μg/ml puromycin (Cellgro).

### Immunofluorescence and Co-Localization

Transfected cells were fixed in 4% paraformaldyhde for 20 minutes at room temperature (RT) then blocked and extracted in 10% normal donkey sera (Jackson ImmunoReaserch), 0.3% Triton X-100 PBS for 30 minutes at RT. For primary antibodies used, see Table 2. All secondary antibodies used were from Jackson ImmunoResearch Lab. Stained cells were mounted with ProLong Gold (Invitrogen). Cells were imaged on a Zeiss LSM 710 confocal microscope using an 63×/1.40 Plan-Apochromat oil immersion lens and 2.5× zoom. Images were processed utilizing Zeiss ZEN blue software and the figures were assembled in Adobe Photoshop. Co-localization analysis was performed on the images using Imaris software (Oxford Instruments). The raw.csz images were convert into the Imaris format with Imaris converter, then opened in the coloc module of Imaris. Thresholds for each image were set utilizing the 2D histogram for the GFP and Cherry channels. The ignore border bins was activated to remove the 0 red and 0 green voxels of the image removing the large number of no fluorescent voxels. Thresholds were set to include only voxels along the diagonal of the 2D histogram and not the bright voxels directly along the X or Y axis. Once the thresholds were set a 3D colocalization channel was created. The calculated data from this channel was exported into excel then imported into Prism to create the Menders' graphs. Differences in Manders' coefficients were evaluated with a Student's t-test. The Zeiss 710 microscope and Imaris software are maintained in the Vanderbilt Cell Imaging Shared Resource.

TABLE 1
Mutagenesis Primers

| Primer Name | Primer Sequence |
|---|---|
| MHVM-E121K | ggtggagcttcaaccccaaaacaaacaacctaatg (SEQ ID NO: 12) |
| MHVM-M126T | aaacaacctaatgtgtacagatacgaaaggtactgtgtatgttagac (SEQ ID |
| | NO: 13) |
| PEDVM-E115K | ggtggtctttcaatcctaaaacagacgcgcttctc (SEQ ID NO: 14) |
| MERSM-E114K | gatcatggtggtcattcaatcctaagactaattgccttttg (SEQ ID NO: 15) |
| CovidM-E115K | tccatgtggtcattcaatccaaaaactaacattcttctcaacg (SEQ ID NO: 16) |

TABLE 2
Antibodies Used

| Antibody | Company | Catalog Number |
|---|---|---|
| GM130 | BD Transduction Labs | 610822 |
| Rab10 | Cell Signaling | 8127P |
| Rab11a | Goldenring Lab | VU57 |
| GFP | Vanderbilt Antibody and | 1C9A5 |
| | Protein Resource | |
| RFP | Rockland | 600-401-379-RTU |
| ERGIC-53 | Proteintech | 13364-1-AP |

### Example 2

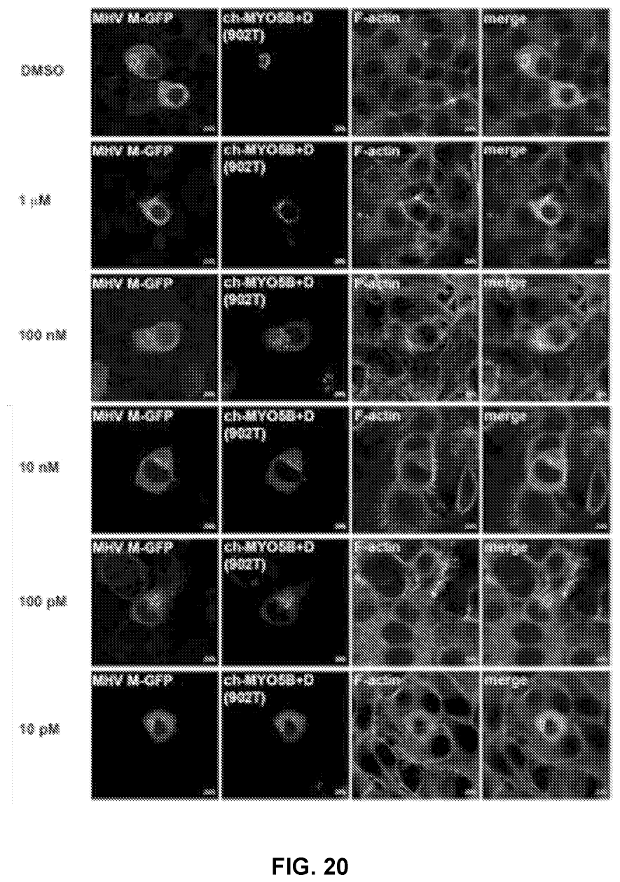

As shown in FIG. 20, Nizatidine disrupts the colocalization of MHV M-GFP with ch-MYO5D+D 902 Tail (902T). A549 cells expressing MHV M-GFP were transfected with mCherry-MYO5B+D 902 Tail and then incubated with Nizatidine at concentrations from 10 pM to 1 μM for 24 hours. Nizatidine cause loss of localization between MHV M and MYO5B+D down to 10 pM. N.B. Cimetidine, Famotidine and Ranitidine at 1 μM had no effect on the co-localization of MHV M with MYO5B+D. Therefore the action seems to be specific compared to three related H2-blockers.

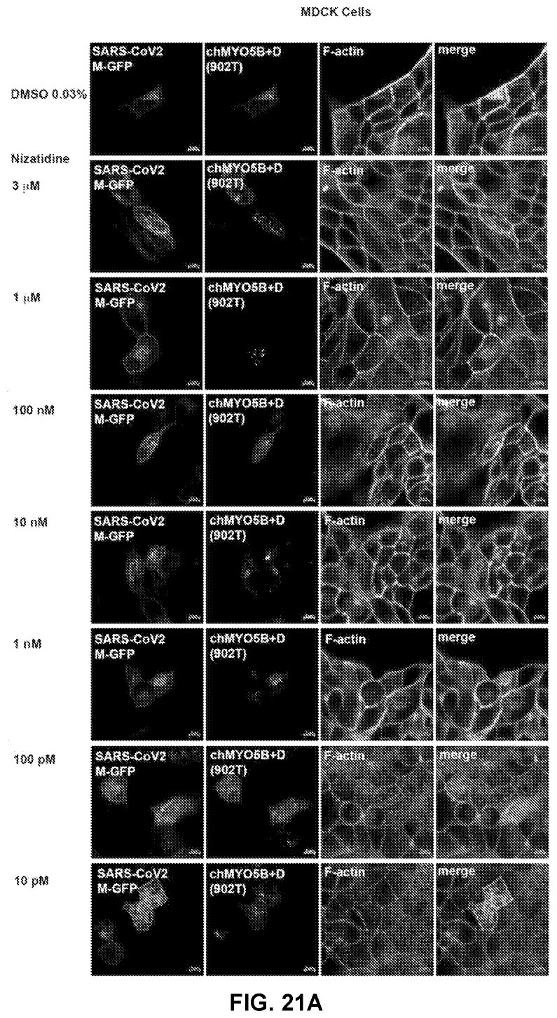

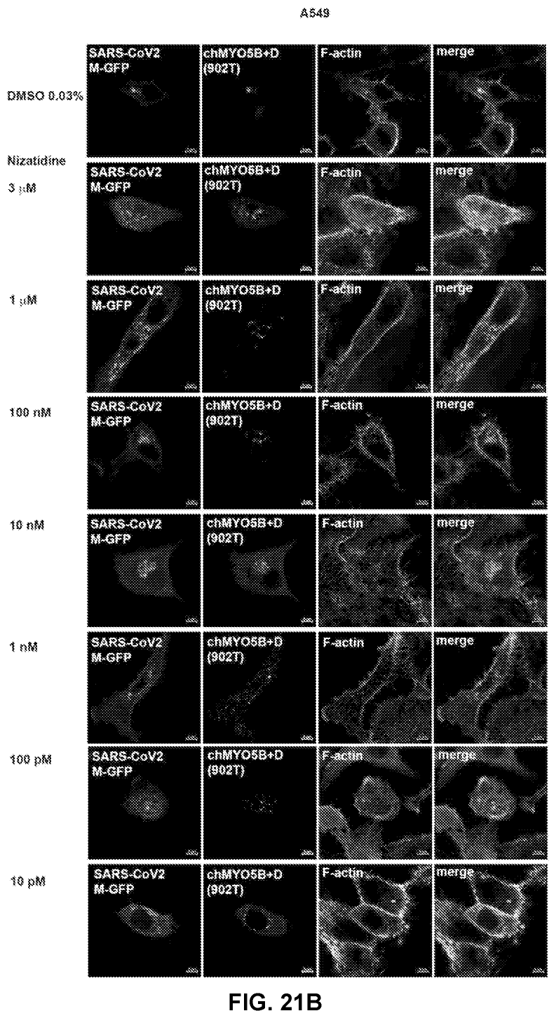

As shown in FIGS. 21A and 21B, Nizatidine disrupts the colocalization of SARS CoV2 M-GFP with ch-MYO5D+D 902 Tail (902T). MDCK cells (FIG. 21A) and A549 cells (FIG. 21B) expressing SARS CoV2 M-GFP were transfected with mCherry-MYO5B+D 902 Tail and then incubated with Nizatidine at concentrations from 10 pM to 1 pM for 24 hours. Nizatidine cause loss of localization between SARS CoV2 M and MYO5B+D down to 10 pM. N.B. Cimetidine, Famotidine and Ranitidine at 1 μM had no effect on the co-localization of SARS CoV2 with MYO5B+D. Therefore the action seems to be specific compared to three related H2-blockers.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for treating or preventing a coronavirus infection in a subject, comprising administering to the subject an effective amount of a composition comprising an agent that disrupts the binding of coronavirus membrane glycoprotein (M protein) and Myosin Vb protein (MYO5B).

2. The method of claim 1, wherein the coronavirus is a betacoronavirus.

3. The method of claim 2, wherein the coronavirus comprises a Mouse Hepatitis Virus (MHV), a Porcine Epidemic Diarrhea Virus (PEDV), a Middle East Respiratory Syndrome virus (MERS-CoV), a severe acute respiratory syndrome virus (SARS-CoV) virus, or a SARS-CoV-2 virus.

4. The method of claim 1, wherein the agent comprises an antibody or aptamer that selectively binds M protein at or near its MYO5B binding site, or selectively binds MYO5B at or near its M protein binding site.

5. The method of claim 1, to wherein the agent comprises a small molecule.

6. The method of claim 1, wherein the agent comprises a soluble fragment of M protein capable of binding human MYO5B.

7. The method of claim 1, wherein the agent comprises a soluble fragment of MYO5B capable of binding M protein.

8. The method of claim 7, wherein the soluble fragment of MYO5B comprises exon D of MYO5B (MYO5B+D).

9. A method for identifying an agent for treating or preventing a betacoronavirus infection, comprising
(a) providing a system comprising coronavirus membrane glycoprotein (M protein) and human Myosin Vb protein (MYO5B) with conditions suitable for binding of the M protein and MYO5B;
(b) contacting the system with a candidate agent; and
(c) assaying the system for binding of M protein and MYO5B
wherein an inhibition in M protein and MYO5B binding is an indication that the agent can be used to treat or prevent a betacoronavirus infection.

10. The method of claim 9, wherein the candidate agent comprises a small molecule.

11. The method of claim 9, wherein the system is a two-hybrid system.


SEQUENCE LISTING (16 sequences)

SEQ ID NO: 1 - protein, 228 aa, Artificial Sequence
  Synthetic Construct
MSSTTQAPEPVYQWTADEAVQFLKEWNFSLGIILLFITIILQFGYTSRSMFIYVVKMIILWLMWPLTIVLCIFNCVYALNNVYLGFSIVFTIVSIVIWIMYFVNSIRLFIRTGSWWSFNPETNNLMCIDMKGTVYVRPIIEDYHTLTATIIRGHLYMQGVKLGTGFSLSDLPAYVTVAKVSHLCTYKRAFLDKVDGVSGFAVYVKSKVGNYRLPSNKPSGADTALLRI

SEQ ID NO: 2 - protein, 104 aa, Artificial Sequence
  Synthetic Construct
SIRLFIRTGSWWSFNPETNNLMCIDMKGTVYVRPIIEDYHTLTATIIRGHLYMQGVKLGTGFSLSDLPAYVTVAKVSHLCTYKRAFLDKVDGVSGFAVYVKSKV

SEQ ID NO: 3 - protein, 219 aa, Artificial Sequence
  Synthetic Construct
MSNMTQLTEAQIIAIIKDWNFAWSLIFLLITIVLQYGYPSRSMTVYVFKMFVLWLLWPSSMALSIFSAVYPIDLASQIISGTVAAVSAMMWISYFVQSIRLFMRTGSWWSFNPETNCLLNVPFGGTTVVRPLVEDSTSVTAVVTNGHLKMAGMHFGACDYDRLPNEVTVAKPNVLIALKMVKRQSYGTNSGVAIYHRYKAGNYRSPPITADIELALLRA

SEQ ID NO: 4 - protein, 122 aa, Artificial Sequence
  Synthetic Construct
SIRLFMRTGSWWSFNPETNCLLNVPFGGTTVVRPLVEDSTSVTAVVTNGHLKMAGMHFGACDYDRLPNEVTVAKPNVLIALKMVKRQSYGTNSGVAIYHRYKAGNYRSPPITADIELALLRA

SEQ ID NO: 5 - protein, 221 aa, Artificial Sequence
  Synthetic Construct
MAENDTITVDQLKHLLEQWNLVIGFLFFAWILLLQFAYSNRNRFLYIIKLVFLWLLWPITLACFVLAAVYRINWATGGIAIAMACLVGLMWLSYFVASFRLFARTRSWWSFNPETNILLNVPLRGSIITRPLLESELVIGAVIIRGYLRMAGHSLGRCDIKDLPKEITVATSRTLSYYRLGASQRVGTDSGFAVYHRYRIGNYKLNTDHSGSNDNIALLVQ

SEQ ID NO: 6 - protein, 124 aa, Artificial Sequence
  Synthetic Construct
SFRLFARTRSWWSFNPETNILLNVPLRGSIITRPLLESELVIGAVIIRGYLRMAGHSLGRCDIKDLPKEITVATSRTLSYYRLGASQRVGTDSGFAVYHRYRIGNYKLNTDHSGSNDNIALLVQ

SEQ ID NO: 7 - protein, 222 aa, Artificial Sequence
  Synthetic Construct
MADSNGTITVEELKKLLEQWNLVIGFLFLTWICLLQFAYANRNRFLYIIKLIFLWLVWPVTLACFVLAAVYRINWITGGIAIAMACLVRLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 8 - protein, 123 aa, Artificial Sequence
  Synthetic Construct
FRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 9 - protein, 1848 aa, Artificial Sequence
  Synthetic Construct
MSVGELYSQCTRVWIPDPDEVWRSAELTKDYKEGDKSLQLRLEDETILEYPIDVQRNQLPFLRNPDILVGENDLTALSYLHEPAVLHNLKVRFLESNHIYTYCGIVLVAINPYEQLPIYGQDVIYTYSGQNMGDMDPHIFAVAEEAYKQMARDEKNQSIIVSGESGAGKTVSAKYAMRYFATVGGSASETNIEEKVLASSPIMEAIGNAKTTRNDNSSRFGKYIQIGFDKRYHIIGANMRTYLLEKSRVVFQADDERNYHIFYQLCAAAGLPEFKELALTSAEDFFYTSQGGDTSIEGVDDAEDFEKTRQAFTLLGVKESHQMSIFKIIASILHLGSVAIQAERDGDSCSISPQDVYLSNFCRLLGVEHSQMEHWLCHRKLVTTSETYVKTMSLQQVINARNALAKHIYAQLFGWIVEHINKALHTSLKQHSFIGVLDIYGFETFEVNSFEQFCINYANEKLQQQFNSHVFKLEQEEYMKEQIPWTLIDFYDNQPCIDLIEAKLGILDLLDEECKVPKGTDQNWAQKLYDRHSSSQHFQKPRMSNTAFIIVHFADKVEYLSDGFLEKNRDTVYEEQINILKASKFPLVADLFHDDKDPVPATTPGKGSSSKISVRSARPPMKVSNKEHKKTVGHQFRTSLHLLMETLNATTPHYVRCIKPNDEKLPFHFDPKRAVQQLRACGVLETIRISAAGYPSRWAYHDFFNRYRVLVKKRELANTDKKAICRSVLENLIKDPDKFQFGRTKIFFRAGQVAYLEKLRADKFRTATIMIQKTVRGWLQKVKYHRLKGATLTLQRYCRGHLARRLAEHLRRIRAAVVLQKHYRMQRARQAYQRVRRAAVVIQAFTRAMFVRRTYRQVLMEHKATTIQKHVRGWMARRHFQRLRDAAIVIQCAFRMLKARRELKALRIEARSAEHLKRLNVGMENKVVQLQRKIDEQNKEFKTLSEQLSVTTSTYTMEVERLKKELVHYQQSPGEDTSLRLQEEVESLRTELQRAHSERKILEDAHSREKDELRKRVADLEQENALLKDEKEQLNNQILCQSKDEFAQNSVKENLMKKELEEERSRYQNLVKEYSQLEQRYDNLRDEMTIIKQTPGHRRNPSNQSSLESDSNYPSISTSEIGDTEDALQQVEEIGLEKAAMDMTVFLKLQKRVRELEQERKKLQVQLEKREQQDSKKVQAEPPQTDIDLDPNADLAYNSLKRQELESENKKLKNDLNELRKAVADQATQNNSSHGSPDSYSLLLNQLKLAHEELEVRKEEVLILRTQIVSADQRRLAGRNAEPNINARSSWPNSEKHVDQEDAIEAYHGVCQTNSKTEDWGYLNEDGELGLAYQGLKQVARLLEAQLQAQSLEHEEEVEHLKAQLEALKEEMDKQQQTFCQTLLLSPEAQVEFGVQQEISRLTNENLDLKELVEKLEKNERKLKKQLKIYMKKAQDLEAAQALAQSERKRHELNRQVTVQRKEKDFQGMLEYHKEDEALLIRNLVTDLKPQMLSGTVPCLPAYILYMCIRHADYTNDDLKVHSLLTSTINGIKKVLKKHNDDFEMTSFWLSNTCRLLHCLKQYSGDEGFMTQNTAKQNEHCLKNFDLTEYRQVLSDLSIQIYQQLIKIAEGVLQPMIVSAMLENESIQGLSGVKPTGYRKRSSSMADGDNSYCLEAIIRQMNAFHTVMCDQGLDPEIILQVFKQLFYMINAVTLNNLLLRKDVCSWSTGMQLRYNISQLEEWLRGRNLHQSGAVQTMEPLIQAAQLLQLKKKTQEDAEAICSLCTSLSTQQIVKILNLYTPLNEFEERVTVAFIRTIQAQLQERNDPQQLLLDAKHMFPVLFPFNPSSLTMDSIHIPACLNLEFLNEV

SEQ ID NO: 10 - protein, 205 aa, Artificial Sequence
  Synthetic Construct
GSPDSYSLLLNQLKLAHEELEVRKEEVLILRTQIVSADQRRLAGRNAEPNINARSSWPNSEKHVDQEDAIEAYHGVCQTNSKTEDWGYLNEDGELGLAYQGLKQVARLLEAQLQAQSLEHEEEVEHLKAQLEALKEEMDKQQQTFCQTLLLSPEAQVEFGVQQEISRLTNENLDLKELVEKLEKNERKLKKQLKIYMKKAQDLEA

SEQ ID NO: 11 - protein, 26 aa, Artificial Sequence
  Synthetic Construct
SKTEDWGYLNEDGELGLAYQGLKQVA

SEQ ID NO: 12 - DNA, 35 bp, Artificial Sequence
  Synthetic Construct
ggtggagcttcaaccccaaaacaaacaacctaatg

SEQ ID NO: 13 - DNA, 47 bp, Artificial Sequence
  Synthetic Construct
aaacaacctaatgtgtacagatacgaaaggtactgtgtatgttagac

SEQ ID NO: 14 - DNA, 35 bp, Artificial Sequence
  Synthetic Construct
ggtggtctttcaatcctaaaacagacgcgcttctc

SEQ ID NO: 15 - DNA, 41 bp, Artificial Sequence
  Synthetic Construct
gatcatggtggtcattcaatcctaagactaattgccttttg

SEQ ID NO: 16 - DNA, 43 bp, Artificial Sequence
  Synthetic Construct
tccatgtggtcattcaatccaaaaactaacattcttctcaacg